(19) United States
(12) Patent Application Publication
(10) Pub. No.: US 2024/0117438 A1
(43) Pub. Date: Apr. 11, 2024

(54) METHOD FOR DETERMINING SUITABILITY TO ANTI-TNF ALPHA THERAPY

(71) Applicants: TECHNION RESEARCH & DEVELOPMENT FOUNDATION LIMITED, Haifa (IL); RAMBAM MED-TECH LTD., Haifa (IL)

(72) Inventors: Shai SHEN-ORR, Haifa (IL); Yehuda CHOWERS, Tel Aviv (IL); Shiran GERASSY-VAINBERG, Haifa (IL); Alexandra BLATT, Shefa Amer (IL); Elina STAROVETSKY, Haifa (IL); Renaud Gilles GAUJOUX, Haifa (IL); Naama MAIMON, Haifa (IL)

(21) Appl. No.: 18/538,027

(22) Filed: Dec. 13, 2023

Related U.S. Application Data
(63) Continuation of application No. PCT/IL2022/050633, filed on Jun. 13, 2022.
(60) Provisional application No. 63/210,023, filed on Jun. 13, 2021.

(51) Int. Cl. C12Q 1/6883; C07K 16/24; G01N 33/564

(52) U.S. Cl. CPC C12Q 1/6883; C07K 16/241; G01N 33/564; C12Q 2600/106; C12Q 2600/158; G01N 2800/52

(57) ABSTRACT

The present invention is directed to a kit and a method for determining the suitability of a subject to a treatment using a tumor necrosis factor alpha (TNFα). Further provided is a method for treating the subject determined to be suitable for a treatment using a TNFα inhibitor.

### CROSS-REFERENCE TO RELATED-APPLICATIONS

This application is a Bypass Continuation of PCT Patent Application No PCT/IL2022/050633 having International filing date of Jun. 13, 2022 which of claims the benefit of priority of U.S. Provisional Patent Application No. 63/210,023, titled “METHOD FOR DETERMINING SUITABILITY TO ANTI-TNF ALPHA THERAPY”, filed Jun. 13, 2021, the contents of which are all incorporated herein by reference in their entirety.

### REFERENCE TO AN ELECTRONIC SEQUENCE LISTING

The contents of the electronic sequence listing (TECH-TRMB-P-0257-US ST26.xml; size: 13,409 bytes; and date of creation: Dec. 13, 2023) is herein incorporated by reference in its entirety.

### FIELD OF THE INVENTION

The present invention, in some embodiments, is in the field of differential diagnosis, and particularly to the determination of suitability or lack thereof, of a subject to anti TNFα therapy.

### BACKGROUND

Inflammatory bowel diseases (IBDs) are characterized by chronic inflammation involving various bowel segments and are associated with an array of extra intestinal manifestations. The etiopathogenesis of IBD is complex and is considered to involve several factors including environmental triggers, microbial dysbiosis, aberrant immune responses and genetic susceptibility. Due to this multifactorial nature and lack of specific mechanistic data, treatments are aimed at controlling the inflammatory process by targeting various immune pathways and cellular populations. Anti-TNFα antibodies are efficient in the treatment of IBD and are thought to exert their effects through several mechanisms, including neutralization of TNFα, induction of cell and complement mediated cytotoxicity through the FC part of the drug, cytokine suppression via reverse signaling or apoptosis and by generation of M2 type monocytes which are thought to suppress inflammation.

Although anti-TNFα has become a staple treatment in IBD, similar to other drugs, a sizable proportion of 30-40% of the treated patients with anti-TNFα monotherapy or even in combination with immune modulators, will present primary non-response to treatment. Previous studies used systematic screening of in-house and meta-analysis data, primarily of intestinal biopsies, for the identification of biomarkers associated with anti-TNFα response failure. Among these, Oncostatin M (OSM) was identified as a potent mucosal biomarker.

The availability of high-resolution molecular data provides opportunities for achieving improved modeling of the complex therapeutic landscape using systems biology and network-based approaches. Yet, most of the statistical methods used are based on population averages, which do not suffice to fully investigate these complex diseases. Although several personalized approaches were recently suggested for exploring sample-level network information, these studies were not cell-centered, and did not decouple cell frequency and cell regulatory program changes. Network structure was used to identify individual alterations in cross-feature relationships between groups, however, these were validated only in the unicellular level. The same is true for the identification of individual-level time series analysis.

### SUMMARY

According to some embodiments, there is provided a method for determining the suitability of a subject to an anti-tumor necrosis factor alpha (TNFα) therapy, and optionally, further administering a therapeutically effective amount of a TNFα inhibitor to a subject determined to be suitable for the anti-TNFα therapy.

The present invention, in some embodiments, is based, at least in part, on the findings of the inventors which identified triggering receptor on myeloid cells 1 (TREM-1) as a predictive biomarker in biopsy and importantly also in blood, which correlated closely with OSM. The identification of these markers implies that analysis of pretreatment immune status is an important tool that may be useful for subject screening for suitability for anti-TNFα therapy.

According to some embodiments, provided herein is a longitudinal cell-centered systems analysis, combining high-dimensional data of whole blood from responding and non-responding patients, at baseline and following two and fourteen weeks post first treatment, which was further validated using real-life cohorts and public datasets. The inventors focused on immune responses in blood, because although presenting analytical challenge due to high background noise, blood-biomarkers have a clear advantage of accessibility and cost-effectiveness.

The present invention, in some embodiments, discloses a single sample-based network approach, termed ‘disruption network’, which provides patient specific hypotheses for lack of response with respect to a global response network.

The present invention, in some embodiments, is based, at least in part, on the findings that monocytic RAC1-PAK1 axis, which is a final common pathway of multiple immunoreceptor signaling cascades is predictive of anti-TNF response in inflammatory bowel disease (IBD) as well as for the same treatment in rheumatoid arthritis (RA).

Therefore, the present invention, in some embodiments, is directed to a unique expression signature that is predictive of a subject's responsiveness or irresponsiveness to anti-TNFα therapy, across immune mediated diseases.

According to one aspect, there is provide a method for determining the suitability of a subject to a treatment with a tumor necrosis factor alpha (TNFα) inhibitor, wherein the TNFα inhibitor is an anti-TNFα antibody or a TNFα mimicking receptor, the method comprising determining an expression level of at least one gene involved in the RAC1-PAK1 axis, in a sample obtained or derived from a subject, wherein an increase in the expression level of the at least one gene involved in the RAC1-PAK1 axis above a pre-determined threshold is indicative of the subject being suitable for treatment with the TNFα inhibitor, thereby determining the suitability of the subject to a treatment with a TNFα inhibitor.

According to another aspect, there is provided a method for treating a subject afflicted with a TNFα related disease, comprising the steps: (a) determining whether an expression level of at least one gene involved in the RAC1-PAK1 axis is increased above a pre-determined threshold, in a sample obtained or derived from the subject; and (b) administering to the subject determined as having increased expression level of at least one gene involved in the RAC1-PAK1 axis above a pre-determined threshold, a therapeutically effective amount of a TNFα inhibitor, wherein the TNFα inhibitor is an anti-TNFα antibody or a TNFα mimicking receptor, thereby treating the subject afflicted with the TNFα related disease.

According to another aspect, there is provided a kit for determining the suitability of a subject for a treatment with a tumor necrosis factor alpha (TNFα) inhibitor, wherein the TNFα inhibitor is an anti-TNFα antibody or a TNFα mimicking receptor, the kit comprising any one of: (a) at least one oligonucleotide comprising a nucleic acid sequence capable of hybridizing to at least one transcript of the at least one gene involved in the RAC1-PAK1 axis, or a complementary DNA thereto; (b) at least one antagonist having specific binding affinity to a protein product of the at least one gene involved in the RAC1-PAK1 axis; and (c) any combination of (a) and (b).

In some embodiments, the at least one gene involved in the RAC1-PAK1 axis is selected from the group consisting of: PAK1, ICAM1, FCGR3A, LYN, IL1B, RAC1, and any combination thereof.

In some embodiments, the TNFα inhibitor is selected from the group consisting of: Infliximab, Adalimumab, Golimumab, Certolizumab pegol, and Etanercept.

In some embodiments, the subject is afflicted with a TNFα related disease selected from the group consisting of: Crohn's disease, ulcerative colitis, rheumatoid arthritis, juvenile idiopathic arthritis, polyarticular juvenile idiopathic arthritis, psoriasis, psoriatic arthritis, plaque psoriasis, ankylosing spondylitis, hidradenitis suppurativa, uveitis and any combination thereof.

In some embodiments, the sample obtained or derived from the subject comprises RNA molecule, protein, or both.

In some embodiments, the any one of the: RNA molecule, protein, and both, is extracted from blood or a biopsy derived or obtained from the subject.

In some embodiments, the determining based on the RNA molecule comprises: hybridization, amplification, sequencing, or any combination thereof, of the RNA molecule.

In some embodiments, the determining based on the protein is by an immune assay comprising an antibody having increased binding affinity to the protein.

In some embodiments, the increase is at least a 10% increase.

In some embodiments, the administering comprises intravenously administering or subcutaneously administering.

In some embodiments, the method further comprises a step (c) comprising at least once determining the expression level of the at least one gene involved in the RAC1-PAK1 axis in the administered subject, wherein an increase in the expression level of the at least one gene involved in the RAC1-PAK1 axis above a pre-determined threshold is indicative of the administered subject being responsive to the treatment with the TNFα inhibitor.

In some embodiments, the TNFα related disease is selected from the group consisting of: Crohn's disease, ulcerative colitis, rheumatoid arthritis, juvenile idiopathic arthritis, polyarticular juvenile idiopathic arthritis, psoriasis, psoriatic arthritis, plaque psoriasis, ankylosing spondylitis, hidradenitis suppurativa, uveitis and any combination thereof.

In some embodiments, the method further comprises a step preceding step (a), comprising extracting RNA, protein, or both, from blood or a biopsy derived or obtained from the subject.

In some embodiments, the at least one oligonucleotide comprises any one of a probing oligonucleotide, a pair of primers capable of amplifying a complementary DNA of the at least one transcript of the at least one gene involved in the RAC1-PAK1 axis in a polymerase chain reaction (PCR), and both.

In some embodiments, the pair of primers is selected from the group consisting of SEQ ID Nos: 1-14.

In some embodiments, the antagonist comprises an antibody.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1D include graphs and illustration showing an external data-driven disease specific molecular response metric, termed herein ‘health axis’, which indicated that responders exhibit a trajectory of treatment-induced immune dynamics while non-responders exhibit an overall opposite direction. (1A) Overview of the ‘health axis’ analysis. (1B) ‘Health axis’ assessment. Left panel, external public (GSE94648) based ‘health axis’ which defines a transition from inflammatory bowel disease (IBD) active disease through inactive disease to healthy state by PCA based differential expressed genes between disease/health states. Right panel, projection of responding patients' samples from our real-life cohort on the ‘health axis’ at the early response period, 2 weeks post first treatment. (1C) Boxplots comparing responders' and non-responders' projection dynamics on the ‘health axis’ at each treatment interval. Statistical significance was calculated by Wilcoxon test. (1D) Scatterplot of the relationship between progress on the ‘health axis’ between baseline to V2 (2 w post first treatment) and between V2 to V3 (14 weeks post first treatment), n=23, Spearman correlation=−0.44, P<0.1).

FIGS. 2A-2C include graphs and a chart showing that normal Infliximab dynamics correlated with changes in monocytes and reduced expression of innate immune related pathways. (2A) Cell frequency alternations following infliximab (IFX) treatment. Left panel, PCA presenting immune cell frequency changes following treatment based on 16 canonical immune populations determined by CyTOF. Arrow tail and head indicate compositional changes at the early (2 w relative to baseline) and later (14 w relative to baseline) response periods correspondingly. Ellipses represent the Euclidean distance from the center. Center panel, scatterplot showing the relationship between changes in monocytes abundance and changes in CRP (n=23, Spearman correlation=0.4, P=0.01). Right panel, boxplots showing change in monocytes abundance following treatment relative to baseline in responders and non-responders. Statistical significance was calculated by Wilcoxon test. (2B) Venn diagram showing dynamic features which significantly changed over time at 2 weeks and 14 weeks post treatment compared with baseline for each response group using linear mixed-effects models (FDR<0.15, n perm for Rand NR: n=1,000 & n=519, respectively). (2C) Scatterplot presenting the normal response network centrality of significantly enriched dynamic pathways at the early response period (fGSEA, FDR<0.25, n perm=1000). Colors indicate pathway median fold change expression at the early response period relative to baseline in responders (colored dots denote significant change in relative pathway score by Wilcoxon test, FDR<0.05).

FIGS. 3A-3E include graphs, illustrations, and heatmaps showing ‘Disruption networks’ as a framework to perform sample level inferences to identify individual variation in drug response. (3A) Disruption network concept, Left panel—a network is generated from a reference group (IFX responders) and then individual subjects from a test group (IFX non-responders) are iteratively added to the obtained response reference network, and the disruption in the correlation structure, defined as a dropout, is assessed for each patient across all edges. Right panel, representative highly disrupted edge demonstrating significant dropout values for non-responders. (3B) Feature specific differential signal between responders and non-responders dynamics at the early response period using disruption measurement of top mean drop intensity (x axis) and standard statistics by Wilcoxon test (y axis). (3C) ‘Disruption networks’ statistic was aggregated across pathways to estimate sample specific disruption in the functional level, according to three parameters including percentage of disrupted edges, mean drop intensity and percentage of disrupted nodes. Top significantly disrupted pathways are presented, defined as those with a complete agreement of all three parameters in the 0.8 percentile. (3D) Distribution of degree and betweenness centrality measurements for nodes belonging to the top disrupted pathways compared to other nodes in the network. Significance was determined using permutation test (n perm=10000). (3E) Meta disrupted pathway. Left panel, response network subgraph consist of nodes from the baseline differential disrupted pathways (FDR<0.1). Dimond shape and orange color represent cell frequency, circle shape represent cell centered expression. Red circles indicate the fiber organization pathway related central axis. Right panel, enrichment analysis of the disrupted pathways by hypergeometric test.

FIGS. 4A-4F include graphs and a heatmap showing the fiber-organization signaling, highly expressed in monocytes, predicts infliximab response at baseline. (4A) Baseline expression differences in the disrupted pathways between response groups by non-parametric multivariate analysis of variance (NPMANOVA; bottom primary axis). Colors denote response network betweenness. The line graph represents correlation of changes in pathway score with changes in CRP (top secondary axis). (4B) The fiber organization differential nodes dynamics assessed by mean relative score across visits for each response group. (4C) Analysis of the cellular origin of the baseline differential fiber organization pathway using scRNA-seq analysis of PBMCs collected from representative responder and non-responder pre-treatment. Left panel, tSNE plot representing cell types identities annotated using singleR based on correlation profiles based on two reference datasets: the Blueprint-Encode (Fernández et al., 2016) and the Monaco Immune Cell (GSE107011; Monaco et al., 2019) datasets. Right panel, tSNE plot colored by the differential fiber organization relative score indicating high expression in monocytes. (4D) The expended fiber organization scaled expression in the different monocyte subsets. The fiber organization baseline differential genes were expended through intersecting knowledge based (stringDB) and data-driven based (Monocyte single cell data) networks. (4E) Mean mTNF expression in the different monocyte subsets as measured by CyTOF. (4F) Baseline prediction performance of IFX response based on the expended fiber organization predictive signature score, in the cell adjusted space. Left panel, receiver operating characteristic (ROC) plots of 100-bootsraps. The predictive signature was determined using elastic net (a=0.5, lambda=0.26, 100 repeated 2-fold CV) based on the baseline differential fiber organization related genes. Significance was determined by permutation test (n perm=1000). Right panel, boxplots of the fiber organization predictive signature score pre-treatment, in the different response groups in the cell-centered bulk expression.

FIGS. 5A-5C include graphs showing validation of the fiber organization predictive signature in an independent IBD cohort and three public Rheumatic arthritis (RA) cohorts pre IFX treatment. (5A) Validation of the pre-treatment predictive fiber organization signature in an additional independent cohort of 20 and 9 responders and non-responders respectively by qPCR. Gene values were normalized to CD14 expression for cell-centered values. Left panel, bar graph of the pre-treatment normalized expression of the signature genes and signature pathway score in each response group. Right panel, receiver operating characteristic (ROC) curve based on the predictive signature relative score. (5B) Prediction performance of fiber organization signaling signature in RA public expression datasets. Left panel, boxplots comparing the fiber organization signature related genes and the pathway score between IFX RA response groups in a representative publicly available dataset GSE20690 (Tanino M. et al., 2010; n=43 responders and 25 non-responders). Right panel, ROC based on the predictive signature relative score of the relevant cohort. (5C) meta-ROC presenting the predictive performance of three independent publicly available RA cohorts: GSE20690 (n=68, of which 43 and 25 are responders and non-responders respectively), GSE33377 (n=42, of which 18 and 24 are responders and non-responders respectively) and GSE42296 (n=19, of which 13 and 6 are responders and non-responders respectively).

FIGS. 6A-6C include graphs showing that CyTOF reveals multiple cell subset changes in responders following treatment and differences between response groups. (6A) Loading plot of PC2 based on major canonical cell composition changes at W2 and W14 compared to baseline. (6B) Cell-type specific alteration in cellular relative abundance during IFX treatment in responders and non-responders (paired-Wilcoxon P-values shown). (6C) Correlation of cell abundance changes at W2 and W14 relative to baseline, with changes in CRP (Spearman's correlation coefficients are shown, P-values are calculated by two tailed probability of the t-statistic, P<0.05 for significant p-values).

FIG. 7 includes graphs showing the cumulative number of discovered dynamic features, at a range of target FDR values by datatype for each response group. Top and bottom panels represent significant changes at W2 and W14 relative to baseline, respectively. FDR was calculated using the Benjamini-Hochberg procedure. Responders were subsampled (n=200) to match the non-responder group size. For responders, average±SEM values are shown.

FIGS. 8A-8D include graphs showing functional pathways associated with IFX response. (8A) Scatterplot of p-values obtained by a comparison of pathway scores between W2 and baseline against those obtained by comparing W14 to baseline (−log10 of paired-Wilcoxon P-values shown). Only globally enriched and network connected pathways were included. (8B) Pathway score related dynamics between W2 and W14 relative to baseline. Top 70 pathways are shown. Pathways are ordered by fold change effect size. P-values for pathway score differences between time points were calculated by paired-Wilcoxon test. Significance was determined by FDR<0.05 (Benjamini-Hochberg procedure). (8C) Heatmap representing a cell-specific contribution for the change in the dynamic pathways. The contribution was determined for each gene in the pathway by regressing its unadjusted fold change expression over the major peripheral cell type frequencies. The reported values represent the mean of the coefficients across all genes in the pathway. (8D) Correlation of pathway score expression with CRP. All time point and response groups are included. (Spearman's correlation coefficients are shown, P-values are calculated by two tailed probability of the t-statistic, Pathway which significantly correlated with CRP (FDR<0.05, Benjamin-Hochberg procedure) are colored.

FIGS. 9A-9C include graphs showing the comparison of the differential signal between response groups dynamics as obtained by the ‘Disruption networks’ framework and standard statistics in the feature level. (9A) Feature specific differential signal between responders and non-responders dynamics at W2 relative to baseline, based on the top disrupted edge ratio (x axis, FDR<0.1 for dropout significance and 10^{th} top percentile of disrupted edge ratio) and standard statistics by Wilcoxon test (y axis, FDR<0.1). (9B) Scatterplot showing feature specific disruption parameters of mean drop intensity against disrupted edge ratio. Points are colored by quartile thresholds (FDR<0.1 for dropout significance and 10^{th} top percentile of the specific disruption parameter). The feature which agreed with the disruption parameters and standard Wilcoxon test is marked with black border. (9C) The cumulative number of disrupted pathways according to each one of the three disruption parameters: mean drop intensity and disrupted edge and node ratio, at a range of top percentile cutoffs. Number of pathways agreed by all parameters in each percentile is also presented. Only pathways that were disrupted in each parameter in at least 60% of the non-responders were included.

FIGS. 10A-10B include heatmaps showing Baseline differences of the significantly dynamics disrupted pathways. (10A) Heatmap representing the feature-level baseline differences among genes in the dynamics meta-disrupted pathway (FDR<0.1, Wilcoxon test). (10B) Correlation between the canonical cellular frequencies as obtained by CyTOF, and the bulk unadjusted expression of the fiber organization related genes in responders (Spearman' s correlation coefficients are shown, P-values are calculated by two tailed probability of the t-statistic). Only significant correlation values are shown (P<0.05 and |r|≥0.5).

FIG. 11 includes a graph showing single cell RNA sequencing (scRNA-seq) based comparison of the baseline fiber organization related expression between the main cell-types and response groups. The fiber organization scaled score based on its baseline differential genes was compared between peripheral blood mononuclear cells (PBMCs) major cell types, and between response groups for monocytes (Wilcoxon P-values shown).

FIG. 12 includes a heatmap representing the top 20 intermediate-monocytes specific enriched pathways associated with the predictive fiber-organization related signature is shown. Pathways were determined by co-expression network based on the pre-treatment expression of the signature predictive genes in intermediate monocyte based on the scRNA-seq data in each response group followed by enrichment analysis (Spearman's correlation, thinning net by 0.1 top percentile, P-adjust<0.05 for functional enrichment significance by hypergeometric test). Pathways displaying significant differences between response groups in each cell subset are colored (FDR<0.05 by Wilcoxon test).

FIGS. 13A-13B include graphs showing differential phosphorylation levels of Ser71-RAC1 in monocytes derived from responders and non-responders, presented as (13A) % in gate, and (13B) median values.

### DETAILED DESCRIPTION

According to some embodiments, there is provided method for determining the suitability of a subject to a treatment using a tumor necrosis factor alpha (TNFα) inhibitor, comprising a step of determining an expression level of at least one gene involved in the RAC1-PAK1 axis, in a sample obtained or derived from a subject.

According to some embodiments, there is provided a method for treating a subject afflicted with a TNFα related disease, comprising the steps: (a) determining whether an expression level of at least one gene involved in the RAC1-PAK1 axis is modified compared to a pre-determined threshold or baseline, in a sample obtained or derived from a subject; and (b) administering to the subject determined as having a modified expression level of the at least one gene involved in the RAC1-PAK1 axis compared to a pre-determined threshold or baseline, a therapeutically effective amount of a TNFα inhibitor.

In some embodiments, a TNFα inhibitor comprises an anti-TNFα antibody. In some embodiments, a TNFα inhibitor comprises a TNFα mimicking receptor. In some embodiments, a TNFα inhibitor comprises a TNFα binding domain. In some embodiments, a TNFα inhibitor comprises a TNFα binding domain of a TNFα receptor or of a mimicking receptor. In some embodiments, a TNFα inhibitor comprises a TNFα soluble receptor. In some embodiments, a TNFα inhibitor comprises a TNFα receptor lacking or being devoid of a transmembrane domain. In some embodiments, a TNFα inhibitor comprises a TNFα receptor being devoid of an intracellular domain or a plurality thereof. In some embodiments, a TNFα inhibitor comprises any protein capable of binding TNFα with an affinity equal to or greater than the endogenous transmembrane (or membrane-anchored) TNFα receptor, as long as it does not permit or enable TNFα signaling or signal transduction. In some embodiments, the binding of TNFα ligand to a TNFα inhibitor as described herein, does not provide TNFα signaling or signal transduction.

The term “TNFα mimicking receptor ” as used herein, is common and would be apparent to one of ordinary skill in the art, and refers to any agent having specific binding affinity to the TNFα ligand, e.g., a TNF receptor binding domain, which is in incapable of propagating TNFα signaling. In some embodiments, TNFα mimicking receptor comprises a TNFα receptor ligand binding domain. In some embodiments, a TNFα mimicking receptor is devoid of a TNFα receptor intracellular domain. In some embodiments, a TNFα mimicking receptor is a soluble agent. In some embodiments, a TNFα mimicking receptor is devoid of an transmembrane domain. In some embodiments, a TNFα mimicking receptor comprises at least one domain or portion of an immunoglobulin. In some embodiments, a TNFα mimicking receptor comprises a constant region of an immunoglobulin. In some embodiments, an immunoglobulin is IgG1.

Non-limiting example a TNFα mimicking receptor, includes, but is not limited to Etanercept, or any functional or biosimilar drug or agent thereof.

In some embodiments, a TNFα mimicking receptor is characterized by being capable of binding a TNFα ligand with essentially the same binding affinity of a TNFα receptor but is incapable of propagating or enabling TNFα signaling.

In some embodiments, modified comprises increased expression level or decreased expression level. In some embodiments, modified comprises upregulated expression or downregulated expression.

According to some embodiments, there is provided a method for treating a subject afflicted with a TNFα related disease, comprising the steps: (a) determining whether an expression level of at least one gene involved in the RAC1-PAK1 axis is increased above a pre-determined threshold or a baseline, in a sample obtained or derived from a subject; and (b) administering to the subject determined as having increased expression level of at least one gene involved in the RAC1-PAK1 axis above a pre-determined threshold or baseline, a therapeutically effective amount of a TNFα inhibitor.

In some embodiments, an increase in the expression level of the at least one gene involved in the RAC1-PAK1 axis above a pre-determined threshold is indicative of the subject being suitable for treatment using a TNFα inhibitor.

In some embodiments, a subject being suitable for treatment using a TNFα inhibitor is defined herein as a “responder” or “responsive”.

In some embodiments, a reduced or decreased expression level of the at least one gene involved in the RAC1-PAK1 axis below a pre-determined threshold is indicative of the subject being unsuitable for treatment using a TNFα inhibitor. In some embodiments, unsuitable comprises first line therapy unsuitability. In some embodiments, a subject determined as unsuitable for first line for treatment using a TNFα inhibitor, may still be treated with a TNFα inhibitor as a second line therapy.

In some embodiments, a subject being unsuitable for treatment using a TNFα inhibitor is defined herein as a “non-responder” or “non-responsive”.

Methods and routes of administering a TNFα inhibitor would be apparent to one of ordinary skill in the art of medicine. Different routes of administration may apply, depending on the inhibitor of choice. At any rate, such route would be apparent to a treating physician given the manufacturer's instructions.

In some embodiments, the method comprises intravenously administering a TNFα inhibitor. In some embodiments, the method comprises subcutaneously administering a TNFα inhibitor.

In some embodiments, the method further comprises a step (c) comprising at least once determining the expression level of the at least one gene involved in the RAC1-PAK1 axis in the administered subject.

In some embodiments, an increase in the expression level of the at least one gene involved in the RAC1-PAK1 axis above a pre-determined threshold is indicative of the administered subject being responsive to the treatment using a TNFα inhibitor.

In some embodiments, the expression output obtained following step (c) indicates whether the administered subject should be further treated with the TNFα inhibitor. In other words, step c is a monitoring step, a confirmation step, a validation step, or any combination thereof, reflecting on the success of the treatment.

Specific genes, proteins, or both, that are involved in the RAC1-PAK1 axis including their sequences, are known in the art of molecular biology and cell biology and would be apparent to a skilled artisan.

In some embodiments, the at least one gene involved in the RAC1-PAK1 axis is selected from: PAK1, LYN, ICAM1, FCGR3A, IL1B, RAC1, or any combination thereof.

In some embodiments, the at least one gene involved in the RAC1-PAK1 axis comprises two genes selected from: PAK1, LYN, ICAM1, FCGR3A, IL1B, and RAC1.

In some embodiments, the at least one gene involved in the RAC1-PAK1 axis comprises three genes selected from: PAK1, LYN, ICAM1, FCGR3A, IL1B, and RAC1.

In some embodiments, the at least one gene involved in the RAC1-PAK1 axis comprises four genes selected from: PAK1, LYN, ICAM1, FCGR3A, IL1B, and RAC1.

In some embodiments, the at least one gene involved in the RAC1-PAK1 axis comprises five genes selected from: PAK1, LYN, ICAM1, FCGR3A, IL1B, and RAC1.

In some embodiments, the at least one gene involved in the RAC1-PAK1 axis comprises: PAK1, LYN, ICAM1, FCGR3A, IL1B, and RAC1.

RAC1 is described in GenBank, see for example, accession number: NM_018890.

In some embodiments, determining the expression level of RAC1 comprises the use of a pair of oligonucleotides comprising the nucleic acid sequences: AGCGGCTGACGTGTGCAGTAAT (SEQ ID NO: 1) and CGAGGGGCTGAGACATTTACAACA (SEQ ID NO: 2).

PAK1 is described in GenBank, see for example, accession number: NM_001376304.1.

In some embodiments, determining the expression level of PAK1 comprises the use of a pair of oligonucleotides comprising the nucleic acid sequences: AGTTTCAGAAGATGAGGATGATGA (SEQ ID NO: 3) and AATCACAGACCGTGTGTATACAG (SEQ ID NO: 4).

LYN is described in GenBank, see for example, accession number: NM_001111097.3.

In some embodiments, determining the expression level of LYN comprises the use of a pair of oligonucleotides comprising the nucleic acid sequences: GCTGGATTTCCTGAAGAGCGATG (SEQ ID NO: 5) and CGGTGAATGTAGTTCTTCCGCTC (SEQ ID NO: 6).

FCGR3A is described in GenBank, see for example, accession number: NM_000569.8.

In some embodiments, determining the expression level of FCGR3A comprises the use of a pair of oligonucleotides comprising the nucleic acid sequences: GGTGACTTGTCCACTCCAGTGT (SEQ ID NO: 7) and ACCATTGAGGCTCCAGGAACAC (SEQ ID NO: 8).

ICAM1 is described in GenBank, see for example, accession number: NM_000201.3.

In some embodiments, determining the expression level of ICAM1 comprises the use of a pair of oligonucleotides comprising the nucleic acid sequences: AGCGGCTGACGTGTGCAGTAAT (SEQ ID NO: 9) and TCTGAGACCTCTGGCTTCGTCA (SEQ ID NO: 10).

IL-1B is described in GenBank, see for example, accession number: NM_000576.3.

In some embodiments, determining the expression level of IL-1B comprises the use of a pair of oligonucleotides comprising the nucleic acid sequences: CCACAGACCTTCCAGGAGAATG (SEQ ID NO: 11) and GTGCAGTTCAGTGATCGTACAGG (SEQ ID NO: 12).

In some embodiments, PAK1 expression increased by at least 5%, at least 15%, at least 25%, at least 35%, at least 50%, at least 75%, at least 100%, at least 150%, at least 250%, at least 500%, at least 750%, or at least 1,000%, compared to a threshold or a baseline, or any value and range therebetween, is indicative of the subject being suitable for anti TNF therapy, as disclosed herein. Each possibility represents a separate embodiment of the invention.

In some embodiments, PAK1 expression increased by 5-50%, 15-200%, 25-450%, 35-600%, 50-500%, 75-700%, 100-1,000%, 150-450%, 250-900%, 500-1,200%, or 10-1,000%, compared to a threshold or a baseline, or any value and range therebetween, is indicative of the subject being suitable for anti TNF therapy, as disclosed herein. Each possibility represents a separate embodiment of the invention.

In some embodiments, a subject determined as being suitable for treatment according to the herein disclose method (e.g., a “responder”) is characterized by having 1.5-3-fold expression of PAK1 compared to a pre-determined threshold or baseline. In some embodiments, a subject determined as being suitable for treatment according to the herein disclose method (e.g., a “responder”) is characterized by having about 2-fold expression of PAK1 compared to a pre-determined threshold or baseline.

In some embodiments, LYN expression increased by at least 5%, at least 15%, at least 25%, at least 35%, at least 50%, at least 75%, at least 100%, at least 150%, at least 250%, at least 500%, at least 750%, or at least 1,000%, compared to a threshold or a baseline, or any value and range therebetween, is indicative of the subject being suitable for anti TNF therapy, as disclosed herein. Each possibility represents a separate embodiment of the invention.

In some embodiments, LYN expression increased by 5-50%, 15-200%, 25-450%, 35-600%, 50-500%, 75-700%, 100-1,000%, 150-450%, 250-900%, 500-1,200%, or 10-1,000%, compared to a threshold or a baseline, or any value and range therebetween, is indicative of the subject being suitable for anti TNF therapy, as disclosed herein. Each possibility represents a separate embodiment of the invention.

In some embodiments, a subject determined as being suitable for treatment according to the herein disclose method (e.g., a “responder”) is characterized by having 1.3-2-fold expression of LYN compared to a pre-determined threshold or baseline. In some embodiments, a subject determined as being suitable for treatment according to the herein disclose method (e.g., a “responder”) is characterized by having about 1.4-fold expression of LYN compared to a pre-determined threshold or baseline.

In some embodiments, ICAM1 expression increased by at least 5%, at least 15%, at least 25%, at least 35%, at least 50%, at least 75%, at least 100%, at least 150%, at least 250%, at least 500%, at least 750%, or at least 1,000%, compared to a threshold or a baseline, or any value and range therebetween, is indicative of the subject being suitable for anti TNF therapy, as disclosed herein. Each possibility represents a separate embodiment of the invention.

In some embodiments, ICAM1 expression increased by 5-50%, 15-200%, 25-450%, 35-600%, 50-500%, 75-700%, 100-1,000%, 150-450%, 250-900%, 500-1,200%, or 10-1,000%, compared to a threshold or a baseline, or any value and range therebetween, is indicative of the subject being suitable for anti TNF therapy, as disclosed herein. Each possibility represents a separate embodiment of the invention.

In some embodiments, a subject determined as being suitable for treatment according to the herein disclose method (e.g., a “responder”) is characterized by having 1.5-3-fold expression of ICAM1 compared to a pre-determined threshold or baseline. In some embodiments, a subject determined as being suitable for treatment according to the herein disclose method (e.g., a “responder”) is characterized by having about 2-fold expression of ICAM1 compared to a pre-determined threshold or baseline.

In some embodiments, FCGR3 expression increased by at least 5%, at least 15%, at least 25%, at least 35%, at least 50%, at least 75%, at least 100%, at least 150%, at least 250%, at least 500%, at least 750%, or at least 1,000%, compared to a threshold or a baseline, or any value and range therebetween, is indicative of the subject being suitable for anti TNF therapy, as disclosed herein. Each possibility represents a separate embodiment of the invention.

In some embodiments, FCGR3 expression increased by 5-50%, 15-200%, 25-450%, 35-600%, 50-500%, 75-700%, 100-1,000%, 150-450%, 250-900%, 500-1,200%, or 10-1,000%, compared to a threshold or a baseline, or any value and range therebetween, is indicative of the subject being suitable for anti TNF therapy, as disclosed herein. Each possibility represents a separate embodiment of the invention.

In some embodiments, a subject determined as being suitable for treatment according to the herein disclose method (e.g., a “responder”) is characterized by having 1.4-3-fold expression of FCGR3 compared to a pre-determined threshold or baseline. In some embodiments, a subject determined as being suitable for treatment according to the herein disclose method (e.g., a “responder”) is characterized by having about 1.5-fold expression of FCGR3 compared to a pre-determined threshold or baseline.

In some embodiments, IL1B expression increased by at least 5%, at least 15%, at least 25%, at least 35%, at least 50%, at least 75%, at least 100%, at least 150%, at least 250%, at least 500%, at least 750%, or at least 1,000%, compared to a threshold or a baseline, or any value and range therebetween, is indicative of the subject being suitable for anti TNF therapy, as disclosed herein. Each possibility represents a separate embodiment of the invention.

In some embodiments, IL1B expression increased by 5-50%, 15-200%, 25-450%, 35-600%, 50-500%, 75-700%, 100-1,000%, 150-450%, 250-900%, 500-1,200%, or 10-1,000%, compared to a threshold or a baseline, or any value and range therebetween, is indicative of the subject being suitable for anti TNF therapy, as disclosed herein. Each possibility represents a separate embodiment of the invention.

In some embodiments, a subject determined as being suitable for treatment according to the herein disclose method (e.g., a “responder”) is characterized by having 1.1-2-fold expression of IL1B compared to a pre-determined threshold or baseline. In some embodiments, a subject determined as being suitable for treatment according to the herein disclose method (e.g., a “responder”) is characterized by having about 1.15-fold expression of IL1B compared to a pre-determined threshold or baseline.

In some embodiments, RAC1 expression increased by at least 5%, at least 15%, at least 25%, at least 35%, at least 50%, at least 75%, at least 100%, at least 150%, at least 250%, at least 500%, at least 750%, or at least 1,000%, compared to a threshold or a baseline, or any value and range therebetween, is indicative of the subject being suitable for anti TNF therapy, as disclosed herein. Each possibility represents a separate embodiment of the invention.

In some embodiments, RAC1 expression increased by 5-50%, 15-200%, 25-450%, 35-600%, 50-500%, 75-700%, 100-1,000%, 150-450%, 250-900%, 500-1,200%, or 10-1,000%, compared to a threshold or a baseline, or any value and range therebetween, is indicative of the subject being suitable for anti TNF therapy, as disclosed herein. Each possibility represents a separate embodiment of the invention.

In some embodiments, a subject determined as being suitable for treatment according to the herein disclose method (e.g., a “responder”) is characterized by having 1.8-3.5-fold expression of RAC1 compared to a pre-determined threshold or baseline. In some embodiments, a subject determined as being suitable for treatment according to the herein disclose method (e.g., a “responder”) is characterized by having about 2-fold expression of RAC1 compared to a pre-determined threshold or baseline.

The terms “pre-determined threshold” or “baseline” are used herein interchangeably and refer to an expression reference point or a control. In some embodiments, the pre-determined threshold or baseline comprise the expression level of at least one gene involved in the RAC1-PAK1 axis being selected from: PAK1, LYN, ICAM1, FCGR3A, IL1B, RAC1, or any combination thereof, in a healthy subject, or in a sample derived or obtained therefrom. In some embodiments, the pre-determined threshold or baseline comprise the expression level of at least one gene involved in the RAC1-PAK1 axis being selected from: PAK1, LYN, ICAM1, FCGR3A, IL1B, RAC1, or any combination thereof, in a TNFα therapy non-responding subject, or in a sample derived or obtained therefrom. In some embodiments, the pre-determined threshold or baseline comprise the expression level of at least one gene involved in the RAC1-PAK1 axis being selected from: PAK1, LYN, ICAM1, FCGR3A, IL1B, RAC1, or any combination thereof, in a subject determined as being non-responding to a TNFα therapy, or in a sample derived or obtained therefrom.

In some embodiments, a TNFα inhibitor is selected from: Infliximab (CAS No. 170277-31-3), Adalimumab (CAS No. 331731-18-1), Golimumab (CAS No. 476181-74-5), Certolizumab pegol (CAS No. 428863-50-7), and Etanercept (CAS No. 185243-69-0).

As used herein, the term “TNFα related disease” refers to any disease, condition, disorder, pathology, or any combination thereof, wherein TNFα is involved, induces, initiates, propagates, determines, or any combination or equivalent thereof, in the pathogenesis, pathophysiology, or both.

In some embodiments, a TNFα related disease is selected from: Crohn's disease, ulcerative colitis, rheumatoid arthritis, psoriasis, or psoriatic arthritis.

In some embodiments, a sample comprises RNA, a protein, or both, derived from a subject. In some embodiments, a protein is a phosphorylated protein.

As used herein, the terms “protein”, “peptide”, and “polypeptide” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the method comprises determining the amount of a phosphorylated protein product of the at least one gene involved in the RAC1-PAK1 axis.

In some embodiments, a reduced amount of a phosphorylated protein product of the at least one gene involved in the RAC1-PAK1 axis below a pre-determined threshold is indicative of the subject being suitable for treatment using a TNFα inhibitor.

In some embodiments, the phosphorylated protein product is phosphorylated on a Serine residue. In some embodiments, the phosphorylated protein product comprises a phosphorylated Serine residue. In some embodiments, the phosphorylated protein product comprises a phosphor-Serine residue. In some embodiments, the protein product is phosphorylated by Protein kinase B/Akt.

In some embodiments, the phosphorylated protein product is RAC1. In some embodiments, the phosphorylated RAC1 protein product comprises a phosphorylated Serine residue at position 71 (e.g., RAC1-Ser71).

In some embodiments, the amount of phosphorylated protein product indicative of the suitability of a subject to anti TNFα therapy, as disclosed herein, is determined in a monocytic cell (e.g., a monocyte) obtained or derived from a subject. In some embodiments, the monocyte is a classic monocyte. In some embodiments, the monocyte is an intermediate monocyte.

In some embodiments, an amount of a phosphorylated protein product reduced by at least 1%, at least 2%, at least 3%, at least 4%, at least 5%, at least 6%, at least 7%, at least 8%, at least 9%, at least 10%, at least 15%, or at least 20%, compared to a threshold or a baseline, or any value and range therebetween, is indicative of the subject being suitable for anti TNF therapy, as disclosed herein. Each possibility represents a separate embodiment of the invention.

In some embodiments, an amount of a phosphorylated protein product reduced by 1-20%, 1-2%, 1-5%, 2-7%, 3-10%, 4-9%, 5-8%, 1-10%, or 2-15%, compared to a threshold or a baseline, or any value and range therebetween, is indicative of the subject being suitable for anti TNF therapy, as disclosed herein. Each possibility represents a separate embodiment of the invention.

In some embodiments, any one of the RNA, protein, and both, is extracted from blood or a biopsy derived or obtain from a subject.

In some embodiments, a biopsy is obtained or derived from a gastrointestinal tract of a subject. In some embodiments, a biopsy comprises an intestinal tissue, cell, any fragment thereof, or any combination thereof.

In some embodiments, the method further comprises a step preceding step (a), comprising extracting RNA from blood of a subject.

Methods for collecting or obtaining a biopsy, blood or any fraction thereof, e.g., PBMCs are common, and would be apparent to one of ordinary skill in the art, such as exemplified herein. Methods for RNA and/or protein extraction, precipitation, purification, or any combination thereof, would also be clear for a person of ordinary skill in the art of molecular biology. Non-limiting example of such RNA extraction methods include, but are not limited to, phenol:chloroform (optionally with iso-amyl alcohol) extraction, followed by ethanol precipitation. Non-limiting examples for extraction and/or protein purification include, but are not limited to, ammonium sulfate precipitation, centrifugation (e.g., ultracentrifugation) with or without a gradient (e.g., sucrose), and chromatography (e.g., size exclusion, affinity, etc.).

In some embodiments, determining comprises nucleic acid amplification reaction. In some embodiments, determining comprises utilization of a polymerase chain reaction (PCR). In some embodiments, determining comprises a quantitative real-time reverse transcription (RT)-PCR. In some embodiments, a quantitative real time RT-PCR comprises relative real time RT-PCR or absolute real time RT-PCR.

In some embodiments, determining comprises nucleic acid hybridization. In some embodiments, hybridization is hybridization with a probing agent. In one embodiment, determining comprises determining by microarray. In one embodiment, determining comprises determining by sequencing. In one embodiment, sequencing comprises next generation sequencing.

In some embodiments, determining comprises specific quantification of a protein as disclosed herein. In some embodiments, determining comprises contacting a sample comprising a protein as disclosed herein with an antibody having increased binding affinity thereto. In some embodiments, the antibody has increased binging affinity to the protein as disclosed herein in a phosphorylated state. In some embodiments, the antibody has increased binging affinity to a phosphorylated form of the protein as disclosed herein.

Methods for determining the amount or expression level of a protein are common and would be apparent to one of ordinary skill in the art of biochemistry and cell biology. Non-limiting examples for such methods include, but are not limited to, western blotting, dot blot, densitometry, enzyme-linked immunosorbent assay (ELISA), radioimmunoassay (RIA), CyTOF, FACS, to name a few.

According to some embodiments, there is provided a composition comprising a TNFα inhibitor for use in the treatment of a subject determined to be suitable for anti TNFα therapy according to the herein disclosed method.

In some embodiments, the composition is a pharmaceutical composition.

### Kit

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

According to some embodiments, there is provided a kit for quantifying expression levels of at least one gene involved in the RAC1-PAK1 axis, comprising any one of: (a) at least one oligonucleotide comprising a nucleic acid sequence capable of hybridizing to at least one transcript of the at least one gene involved in the RAC1-PAK1 axis, or a complementary DNA thereto; (b) at least one antagonist having specific binding affinity to a protein product of the at least one gene involved in the RAC1-PAK1 axis; and (c) a combination of (a) and (b).

In some embodiments, the kit is for determining the suitability of a subject for a treatment with a tumor necrosis factor alpha (TNFα) inhibitor, wherein the TNFα inhibitor is an anti-TNFα antibody or a TNFα mimicking receptor.

In some embodiments, the at least one oligonucleotide comprises any one of a probing oligonucleotide and a pair of primers capable of amplifying a complementary DNA of the at least one transcript of the at least one gene involved in the RAC1-PAK1 axis in a polymerase chain reaction (PCR).

In some embodiments, the antagonist comprises an antibody.

In one embodiment, the present invention provides a kit for amplifying a cDNA molecule of a transcript of a gene involved in the RAC1-PAK1 axis. In one embodiment, the present invention provides a kit for quantifying the amount of a cDNA molecule of a transcript of a gene involved in the RAC1-PAK1 axis. In some embodiments, the kit comprises DNA primers or oligonucleotides for amplifying the nucleic acid molecule (e.g., cDNA molecule of a transcript of a gene involved in the RAC1-PAK1 axis) as described herein in a polymerase chain reaction (PCR). In some embodiments, the present invention provides a kit for extracting RNA from a sample derived or obtained from a subject, such as, but not limited to a blood sample, e.g., comprising PBMC. In some embodiments, the present invention provides a kit for reverse transcribing a messenger RNA (mRNA) or a plurality thereof, to complementary DNA (cDNA) or a plurality thereof. In some embodiments, mRNA comprises total mRNA.

As used herein, the term “total mRNA” refers to a composition representing all mRNA transcripts isolated or extracted from a biological sample, as described herein.

In some embodiments, the kit comprises instruction for RNA extraction, reverse transcription, or both.

In one embodiment, PCR comprises denaturing double-stranded DNA in a sample (to separate the complementary strands), annealing the primers to the dissociated DNA strands, and extension reaction from the primers catalyzed by a thermostable DNA polymerase, the cycle is then repeated.

In one embodiment, a pair of DNA primers as described herein are specifically complementary to and hybridizing with opposite strands DNA with one to the left (5′) and one to the right (3′) of the target sequence within the cDNA molecule of a transcript of a gene involved in the RAC1-PAK1 axis to be amplified. In one embodiment, the target sequence within the cDNA molecule of a transcript of a gene involved in the RAC1-PAK1 axis to be amplified is the nucleic acid molecule as described herein. In one embodiment, the presence of a nucleic acid molecule as described herein (e.g., cDNA molecule of a transcript of a gene involved in the RAC1-PAK1 axis) in an amount above a pre-determined threshold or baseline as described herein, in a sample derived or obtained from a subject, provides direct evidence for the suitability of the subject to an anti-TNFα therapy. In one embodiment, the presence of a nucleic acid molecule as described herein (e.g., cDNA molecule of a transcript of a gene involved in the RAC1-PAK1 axis) in an amount below a pre-determined threshold or baseline as described herein, in a sample derived or obtained from a subject, provides direct evidence for the unsuitability of the subject to an anti-TNFα therapy.

In one embodiment, a kit as described herein further comprises a Reverse Transcriptase. In one embodiment, a kit as described herein further comprises a DNA polymerase. In one embodiment, a kit as described herein further comprises a thermostable DNA polymerase.

In one embodiment, a kit as described herein comprise a PCR buffer. In one embodiment, a PCR buffer comprises: 5 to 100 mM Tris-HCl and 20 to 100 mM KCl. In one embodiment, a PCR buffer further comprises 10 to 100 mM Magnesium chloride. In one embodiment, a kit as described herein comprise a dNTP mixture. In one embodiment, a kit as described herein comprises RNA extracted from a cell. In one embodiment, a kit as described herein comprises total RNA extracted from a cell. In one embodiment, a kit as described herein comprise DNA Polymerase such as but not limited to Taq DNA Polymerase. In one embodiment, a kit as described herein comprise distilled water.

In some embodiments, the kit comprises primers or oligonucleotides suitable for amplifying the RAC1 gene (GenBank, accession number: NM_018890), or a fragment thereof.

In some embodiments, the kit comprises a pair of oligonucleotides comprising the nucleic acid sequences: AGCGGCTGACGTGTGCAGTAAT (SEQ ID NO: 1) and CGAGGGGCTGAGACATTTACAACA (SEQ ID NO: 2).

In some embodiments, the kit comprises primers or oligonucleotides suitable for amplifying the PAK1 gene (GenBank, accession number: NM_001376304.1), or a fragment thereof.

In some embodiments, the kit comprises a pair of oligonucleotides comprising the nucleic acid sequences: AGTTTCAGAAGATGAGGATGATGA (SEQ ID NO: 3) and AATCACAGACCGTGTGTATACAG (SEQ ID NO: 4).

In some embodiments, the kit comprises primers or oligonucleotides suitable for amplifying the LYN (GenBank, accession number: NM_001111097.3), or a fragment thereof.

In some embodiments, the kit comprises a pair of oligonucleotides comprising the nucleic acid sequences: GCTGGATTTCCTGAAGAGCGATG (SEQ ID NO: 5) and CGGTGAATGTAGTTCTTCCGCTC (SEQ ID NO: 6).

In some embodiments, the kit comprises primers or oligonucleotides suitable for amplifying the FCGR3A gene (GenBank, accession number: NM_000569.8), or a fragment thereof.

In some embodiments, the kit comprises a pair of oligonucleotides comprising the nucleic acid sequences: GGTGACTTGTCCACTCCAGTGT (SEQ ID NO: 7) and ACCATTGAGGCTCCAGGAACAC (SEQ ID NO: 8).

In some embodiments, the kit comprises primers or oligonucleotides suitable for amplifying the ICAM1 gene (GenBank, accession number: NM_000201.3), or a fragment thereof.

In some embodiments, the kit comprises a pair of oligonucleotides comprising the nucleic acid sequences: AGCGGCTGACGTGTGCAGTAAT (SEQ ID NO: 9) and TCTGAGACCTCTGGCTTCGTCA (SEQ ID NO: 10).

In some embodiments, the kit comprises primers or oligonucleotides suitable for amplifying the IL-1B gene (GenBank, accession number: NM_000576.3), or a fragment thereof.

In some embodiments, the kit comprises a pair of oligonucleotides comprising the nucleic acid sequences: CCACAGACCTTCCAGGAGAATG (SEQ ID NO: 11) and GTGCAGTTCAGTGATCGTACAGG (SEQ ID NO: 12).

In some embodiments, the kit comprises a plurality of oligonucleotides or primer pairs, wherein the primer pairs are suitable for amplification of PAK1, LYN, ICAM1, FCGR3A, IL1B, and RAC1.

In some embodiments, the kit comprises SEQ ID Nos.: 1-14.

In some embodiments, the kit comprises one or more oligonucleotides capable of hybridizing to any of SEQ ID Nos.: 1-14. In some embodiments, the one or more oligonucleotides capable of hybridizing to any of SEQ ID Nos.: 1-14, are probes. In some embodiments, the one or more oligonucleotides capable of hybridizing to any of SEQ ID Nos.: 1-14 comprises a detectable moiety. In some embodiments, the one or more oligonucleotides capable of hybridizing to any of SEQ ID Nos.: 1-14 are labeled.

In some embodiments, the kit comprises instructions for contacting a sample with one or more oligonucleotides capable of hybridizing to any of SEQ ID Nos.: 1-14 each having specific affinity to one gene involved in the RAC1-PAK1 axis, and determining the expression level of the one or more genes involved in the RAC1-PAK1 axis in the sample.

In some embodiments, determining comprises detecting a signal indicative of the hybridization of the one or more oligonucleotides capable of hybridizing to any of SEQ ID Nos.: 1-14. In some embodiments, hybridization comprises base pairing of the one or more oligonucleotides capable of hybridizing to any of SEQ ID Nos.: 1-14 and complementary polynucleotides comprised by the sample. In some embodiments, the complementary polynucleotides comprised by the sample comprises DNA and/or RNA polynucleotides.

In some embodiments, the signal indicative of the hybridization comprises any one of: a fluorescent signal, a radioactive signal, and a chromatic signal.

In some embodiments, the one or more oligonucleotides capable of hybridizing to any of SEQ ID Nos.: 1-14 is any one of: fluorescently labeled, radioactively labeled, and chromatically labeled. In some embodiments, the one or more oligonucleotides capable of hybridizing to any of SEQ ID Nos.: 1-14 comprises a molecule or a moiety embedded or incorporated therein. In some embodiments, the molecule or moiety are further recognized and/or bound by a molecule having increased binding affinity to the molecule or moiety, such as a specific antibody (e.g., digoxigenin (DIG) and an anti-DIG antibody) or a binding counterpart (e.g., avidin and biotin). In some embodiments, the antibody or binding counterpart is further linked to an enzyme. In some embodiments, the linked enzyme is capable of catalyzing colorimetric reaction. In some embodiments, the colorimetric reaction comprises a bioluminescent reaction or a chemiluminescent reaction.

In some embodiments, the kit comprises instruction for amplifying PAK1, LYN, ICAM1, FCGR3A, IL1B, and RAC1, so as to determine the suitability of a subject to an anti TNF therapy, as described herein. In some embodiments, the kit comprises instruction for quantifying the expression level of PAK1, LYN, ICAM1, FCGR3A, IL1B, and RAC1, so as to determine the suitability of a subject to an anti TNF therapy, as described herein.

In some embodiments, the kit comprises a pair of oligonucleotides suitable for the amplification of a monocyte specific marker. Monocyte specific markers including their sequence, and source, would be apparent to one of skill in the art.

In some embodiments, the monocyte specific marker is used as a normalizing agent for determining the expression level of a gene as disclosed herein.

In some embodiments, the kit further comprises instructions for normalizing the expression of a gene as disclosed herein based on or in reference to a monocyte specific marker.

In some embodiments, the monocyte marker comprises CD14.

In some embodiments, the kit comprises primers or oligonucleotides suitable for amplifying the CD14 (GenBank, accession number: KJ890855.1), or a fragment thereof.

In some embodiments, the kit comprises a pair of oligonucleotides comprising the nucleic acid sequences: CTGGAACAGGTGCCTAAAGGAC (SEQ ID NO: 13) and GTCCAGTGTCAGGTTATCCACC (SEQ ID NO: 14).

In some embodiments, the kit comprises an antagonist having specific binding affinity to a protein product of a gene involved in the RAC1-PAK1 axis. In some embodiments, the antagonist has specific binding affinity to a phosphorylated protein product of a gene involved in the RAC1-PAK1 axis. In some embodiments, the antagonist is selected from: an antibody, a mimicking receptor, a binding domain, a binding domain of a mimicking receptor, a soluble receptor, a receptor lacking or being devoid of a transmembrane domain, or a receptor being devoid of an intracellular domain or a plurality thereof.

In some embodiments, the kit further comprises instruction for contacting a sample with at least one antagonist as described herein, thereby determining the amount of one or more genes involved in the RAC1-PAK1 axis, as described herein.

Methods for using antagonists, e.g., antibodies, for determination and/or quantification of a ligand molecule of the antagonist, are common and would be apparent to one of ordinary skill in the art. Non-limiting examples of such methods include, but are not limited to, ELISA, dot blot, western-blot, immunoprecipitation, densitometry, their combinations, as well as others, some of which are exemplified herein.

The terms “gene” and “transcript” are used herein interchangeably and refer to a nucleic acid sequence of a gene (DNA) or its transcription product (“transcript”), wherein the gene/transcript is involved in the RAC1-PAK1 axis, as described herein.

In some embodiments, the kit as described herein further comprises a TNFα inhibitor. In some embodiments, the kit as described herein comprises a plurality of TNFα inhibitors. In some embodiments, the kit comprises a first TNFα inhibitor selected from: Infliximab (CAS No. 170277-31-3), Adalimumab (CAS No. 331731-18-1), Golimumab (CAS No. 476181-74-5), Certolizumab pegol (CAS No. 428863-50-7), and Etanercept (CAS No. 185243-69-0).

As used herein, the term “plurality” refers to any integer equal to or greater than 2.

In some embodiments, the kit further comprises instructions for administering a first TNFα inhibitor to a subject determined to be as suitable for treatment using a first TNFα inhibitor (e.g., a “responder” or “responding” subject, as described herein).

In some embodiments, the kit further comprises a second TNFα inhibitor. In some embodiments, the first TNFα inhibitor and the second TNFα inhibitor are not the same. In some embodiments, the second TNFα does not include: Infliximab (CAS No. 170277-31-3), Adalimumab (CAS No. 331731-18-1), Golimumab (CAS No. 476181-74-5), Certolizumab pegol (CAS No. 428863-50-7), and Etanercept (CAS No. 185243-69-0).

In some embodiments, the kit further comprises instruction for administering a second TNFα inhibitor to a subject determined to be as unsuitable for treatment using a first TNFα inhibitor (e.g., a “non-responder” or “non-responding” subject, as described herein).

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value (±10%). For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Patients and Study Design

### Patient in the Primary Real-Life Cohort

The primary real-life cohort included 24 Crohn's disease (CD) patients who received Infliximab (IFX) anti-tumor necrosis factor alpha (TNFα) treatment at the gastroenterology department of the Rambam Health Care Campus (RHCC) and met the study inclusion criteria as follows: (1) Adequately documented active luminal CD, as diagnosed by a gastroenterologist with expertise in inflammatory bowel disease (IBD). (2) Documented decision to initiate full Infliximab induction regimen with 5 mg/kg induction dosing (e.g., at weeks 0, 2, 6). Patients that had past exposure to Infliximab, Adalimumab or Vedolizumab, or patients who had active infection including febrile diseases or intra-abdominal or perianal abscess were excluded. The study was approved by the institutional review board, and patients provided written informed consent (https://www.biorxiv.org/content/10.1101/2021.06.16.448558v1.supplementary-material?versioned=true).

Patient samples were obtained at three time points: at baseline, before Infliximab treatment, and two and fourteen weeks post first treatment and assayed for gene expression microarray data, high-resolution granulocytes and lymphocytes subtype frequencies and functional markers by CyTOF, and a panel of 51 cytokines and chemokines by Luminex. CyTOF panel including Clone, vendor, and conjugation information, and Luminex panel are detailed (biorxiv.org/content/10.1101/2021.06.16.448558v1.supplementary-material?versioned=true).

Patient response classification was defined by decision algorithm, which the inventors used and described previously (Gaujoux, R. et al., 2019). Briefly, patients were classified as responders based on clinical remission, which was defined as cessation of diarrhea and abdominal cramping or, in the cases of patients with fistulas, cessation of fistula drainage and complete closure of all draining fistulas at week 14, coupled with a decision of the treating physician to continue IFX therapy at the current dosing and schedule. Patients that were defined as partial responders, classification was determined by the decision algorithm that included the following hierarchical rules: (1) steroid dependency at week fourteen; (2) biomarker dynamics (Calprotectin and CRP) and 3) response according to clinical state at week 26.

Applying the decision algorithm and exclusion criteria, yielded a final study cohort of 15 and 9 responding and non-responding patients, respectively.

Responders were found to have significantly reduced CRP, already at week 2 post first treatment while non-responders presented a trend of reduced CRP at week 2, but their CRP level following 14 weeks was elevated and significantly higher than CRP level in responders (biorxiv.org/content/10.1101/2021.06.16.448558v1 .supplementary-material?versioned=true). No significant difference was found in target TNFα levels, neither in responders or non-responders, as measured by either serum cytokine level using Luminex or by adjusted gene expression. As expected, IFX drug levels were shown to be significantly reduced, in both responders and non-responders at week 14 compared to week 2, due to the transition from induction to maintenance therapy. Drug levels of responders were significantly higher compared to non-responders at week 14. However, at week 2, drug levels were higher in the non-responders' group as compared to responders. Responders also showed improved albumin levels along treatment, with significantly higher levels compared to non-responders at week 14. All other parameters were comparable between the two response groups.

### Patients in the Validation Real-Life Cohort

The validation cohort included 29 CD patients from the RHCC, which were classified to 20 and 9 clinical responding and non-responding patients (according to the above-described decision algorithm (biorxiv.org/content/10.1101/2021.06.16.448558v1 .supplementary-material?versioned=true).

Whole blood samples were collected from the patients pre-treatment, and PBMCs were isolated using density gradient centrifugation by spinning blood over UNI-SEPmaxi+ tubes (Novamed Ltd.) following the manufacturer's protocol. The isolated cells were resuspended in 1 ml freezing solution, containing 10% DMSO and 90% FCS and kept in Nalgene Mr. Frost® Cryo 1° C. Freezing Container (ThermoFisher scientific) with Isopropyl alcohol at −80° C. over-night, and immediately after, placed in a liquid nitrogen container for long-term storage.

At the stage of RNA extraction, the frozen PBMCs were rapidly thawed at 37° C. and transferred into 50 mL centrifuge tubes. The cryovials were rinsed with 1 mL of warm (37° C.) RPMI 1640 supplemented with 10% of FCS which was added dropwise to the DMSO containing fraction while gently shaking the cells. Next, the cells were gradually diluted by adding 30 ml medium dropwise. The diluted cell suspension was centrifuged for 5 min at 300 g. Most of the supernatant was discarded leaving ˜1 ml, and the cells were resuspended in 9 ml of medium followed by additional centrifugation for 5 min at 300 g for washing. RNA was then extracted using RNeasy mini kit (QIAGEN) according to the manufacturer's instruction. Complementary DNA was synthesized using Maxima first strand cDNA synthesis kit with dsDNase (Thermo Scientific). Quantitative PCR (qPCR) was performed using 7300 Real-Time PCR System (AB Applied Biosystems). Relative cytokine expression was calculated following normalization to glyceraldehyde-3 phosphate dehydrogenase (GAPDH) expression (biorxiv.org/content/10.1101/2021.06.16.448558v1.supplementary-material?versioned=true). Primers were purchased from Sigma Aldrich. The expression of the genes in the predictive signature was calculated relative to CD14 expression, to measure monocytes' centered differential expression between response groups, pre-treatment.

### CyTOF Sample Processing and Analysis

A total of 2×10⁶ cells of each sample were stained (1 h; room temperature) with a mixture of metal-tagged antibodies biorxiv.org/content/10.1101/2021.06.16.448558v1.supplementary-material?versioned=true). This mix contained antibodies against phenotyping markers of the main immune populations and some central cytokine and chemokine receptors. All antibodies were validated by the manufacturers for flow application (as indicated on the manufacturer's datasheet, available online) and were conjugated by using the MAXPAR reagent (Fluidigm Inc.). Iridium intercalators were used to identify live and dead cells. The cells were fixed in 1.6% formaldehyde (Sigma-Aldrich; 1 h) at 4° C. until they were subjected to CyTOF mass cytometry analysis on a CyTOF I machine (Fluidigm Inc.). Cell events were acquired at approximately 500 events/s. To overcome potential differences in machine sensitivity and a decline of marker intensity over time, the inventors spiked each sample with internal metal-isotope bead standards for sample normalization by CyTOF software (Fluidigm Inc.) as previously described (Finck et al., 2013).

For data preprocessing, the acquired data were uploaded to the Cytobank web server (Cytobank Inc.) to exclude dead cells and bead standards. The processed data were analyzed using Citrus algorithm, which performs hierarchical clustering of single cell-events by a set of cell-type defining markers and then assigns per sample, per cluster its relative abundance in each sample as well as the median marker expression for each functional marker per cluster (Bruggner et al., 2014). Citrus analysis was applied separately on PBMCs and Granulocytes population in each sample using the following parameters: minimum cluster size percentage of 0.01 and 0.02 for PBMCs and Granulocytes respectively, subsampling of 15,000 events per sample and arcsin hyperbolic transform cofactor of 5. The gating for the classification of the clusters is detailed (biorxiv.org/content/10.1101/2021.06.16.448558v1.supplementary-material?versioned=true).

### Blood Transcriptome Analysis

Whole blood was maintained in PAXgene Blood RNA tubes (PreAnalytiX). RNA was extracted and assayed using Affymetrix Clariom S chips (Thermo Fisher Scientific). The microarray data are available at the Gene Expression Omnibus database (http://www.ncbi.nlm.nih.gov/geo/) under accession number GSE107865. The raw gene array data were processed to obtain a log2 expression value for each gene probe set using the RMA (robust multichip average) method available in the affy R package. Probe set annotation was performed using affycoretools and clariomshumantranscriptcluster.db packages in R. Data were further adjusted for batch effect using empirical Bayes framework applied by the Combat R package.

Gene expression data were further adjusted for variations in frequency of major cell types across samples as measured by CyTOF, including CD4+ T cells, CD8+ T cells, CD19+ B cells, NK cells, monocytes, and granulocytes, to allow detection of differential biological signals that do not stem from cell proportion differences, which might be otherwise masked in unadjusted gene expression data. Adjustment was performed using the CellMix R package.

### Cytokines and Chemokines Measurement Using Luminex Bead-Based Multiplex Assay

Serum was separated from whole blood specimens and stored at −80° C. until used for cytokine determination. Samples were assayed in duplicate according to the manufacturers' specifications (ProcartaPlex™ Immunoassay, EPX450-12171-901, eBioscience, Cytokine/Chemokine/Growth Factor 45-Plex Human Panel 1; biorxiv.org/content/10.1101/2021.06.16.448558v1.supplementary-material?versioned=true).

Data were collected on a Luminex 200 instrument and analyzed using Analyst 5.1 software (Millipore) and NFI (Median Fluorescence Intensity) values were used for further data processing. A pre-filtering was applied as follows: samples with low mean bead count, below 50 were excluded from analysis. In addition, duplicates with high CV values (Coefficient of variation) above 40% were omitted. NFI values with low bead count, below 20 were filtered out, but in cases which one replicate had acceptable bead count and the CV values for both replicates were less than 25%, NFI values were retained.

Finally, net MFI values were calculated by blank reduction followed by log2 transformation. Data were further adjusted for batch effect using the empirical Bayes framework applied by the Combat R package.

### Characterization of IFX Responders and Non-Responders' Dynamics Through Integrative Molecular Response Axis Combining External and In-House Data

An integrative molecular response axis was constructed to recapitulate the complex nature of anti-TNFα response progression dynamics which enables to track individual immune dynamics of both responding and non-responding patients. This methodology was assessed using in-house data and was also validated using an external data-based axis.

For unbiased definition of the health axis and validation of the herein disclosed data, the inventors used public gene expression data of whole blood from 25 ulcerative colitis (UC) patients and 50 CD patients in active or inactive disease states, available in Gene Expression Omnibus (GSE94648). The patients in this external cohort were treated with different medications including 5-ASAs, immunosuppressants, anti-TNF agents, steroids, and combinations of these therapies, as previously described (Planell et al., 2017), representative of a relatively large portion of the treated IBD patient population. The analysis was performed in several steps: (1) Differential expression analysis between active disease and healthy states for UC and CD separately (biorxiv.org/content/10.1101/2021.06.16.448558v1.supplementary-material?versioned=true), using the limma R package, followed by PCA (Principle Component Analysis); (2) Ordinal lasso was used to select the principle components that best describe the desired directionality from active through inactive to healthy state, based on optimal absolute coefficient values and percentage of variance explained parameters (biorxiv.org/content/10.1101/2021.06.16.448558v1.supplementary-material?versioned=true); (3) The ‘Health axis’ coordinates were defined based on initial and terminal points determined as the mean of the two end-point coordinates of active and healthy states; (4) Applying vector multiplication (dot product) for the calculation of the projection of sample vector from our in-house cohort in the direction of the external ‘health axis’, to estimate sample position on the axis; and (5) Evaluation of the distance of patient samples between two time points based on sample axis location.

### A Multi-Dimensional Network Analysis for the Characterization of Normal Efficient Response Dynamics to Anti-TNF Treatment

To identify features that change over time in responders, a linear mixed-effects model was used, in which time was treated as a fixed effect and individuals were treated as a random effect (lmer R package) to allow testing differential expression by time while accounting for between-subject variations. P-values were calculated empirically through a permutation test. In each permutation, feature measurements were shuffled between visits for each responding patient. Permutation based p-values were obtained by comparing the absolute value of the non-permuted β coefficient for each feature to the null distribution of permuted β coefficients for the same feature. In order to calculate false discovery rate (FDR) based on the permutation results, permuted p-value was determined for each permuted β coefficient, by comparing the tested permuted β coefficient to the distribution of the other permuted β coefficients for each feature. Then FDR was estimated by comparing the non-permuted p-values to the null distribution of the permuted p-values. A similar calculation was performed for non-responders.

In addition to the determination of dynamic features in the full responders' sample data, a random subsampling of samples from the responders group, without replacement, was applied to achieve equal sample size between responders and non-responders. Two-hundred subsamples were generated and tested using linear mixed-effects models. In this part, for the comparison of equally sized responders and non-responders' groups, p-values were calculated based on the t-statistic using the Satterthwaite approximation, implemented in the lmerTest R package, followed by multiple hypotheses correction using the Benjamini-Hochberg procedure.

Co-expression network based on V1-V2 fold-change expression values of the significantly altered features (FDR<0.15) was constructed, based on pairwise Spearman's rank correlation using the psych R package. Filtering was applied to remove feature-pairs with insignificant correlation with a cutoff of FDR<0.1.

Network propagation procedure was applied to enhance the biological signal of the obtained networks as previously described (Li et al., 2014) with slight modifications. Briefly, for each node in the network, protein interactors with a combined score above 700 were extracted based on STRING database (functional protein association networks; https://string-db.org/cgi/download.p1) using STRINGdb R package (Szklarczyk et al., 2015). A node interactor was added as a linker gene to the network if its own interactors (hubs) were significantly enriched in the core network features. Enrichment was calculated using the hypergeometric test in the stats R package. Calculated p-values were adjusted for multiple hypotheses using the Benjamini-Hochberg procedure. A cutoff of FDR<0.05 was selected for significant enrichment of the tested interactor hubs in the immune network.

To assess dynamics in the functional level, genes were grouped to functional sets by using a semi-supervised approach combining both network structure and known gene set annotations from Hallmark, Kegg, Reactome, Biocarta, PID and BP Go terms. Each edge in the network was classified to a specific pathway if its two linked nodes were annotated in the same biological group. Pathways with less than 5 mapped edges were filtered out. This was followed by a global gene set enrichment analysis using fGSEA (FDR<0.15, nperm=1000, minSize=10, maxSize=400; biorxiv.org/content/10.1101/2021.06.16.448558v1.supplementary-material?versioned=true).

The dynamic enriched pathway structures were further tested for significance by comparing the density (graph density score) of each pathway associated sub-network to a parallel sub-network densities obtained from 100 random networks with a matched size according to the Erdos-Renyi model which assigns equal probability to all graphs with identical edge count (igraph R package). P-value was evaluated as the proportion of random module density scores that were higher than the real module density score. Additional filtering was applied according to the number of connected components in a pathway sub-graph (igraph R package). Only highly connected pathways (percentage of largest connected component>50%, size of the connected component>10) were included.

The dynamic pathways list was further condensed by filtering out high overlapping pathways using Jaccard index. Accordingly, in overlapping pathways pairs that presented a Jaccard index above 0.5 the smaller module was omitted.

To further associate the assigned pathways with treatment response, the Wilcoxon test was used to compare V1 to V2 and V1 to V3 scaled pathway scores in responders and non-responders. p-values were adjusted for multiple hypotheses using the Benjamini-Hochberg procedure (FDR<0.05). Pathway scores were calculated for each sample as previously described 76. In short, the expression of each gene in the pathway was standardized by the z-score transformation, to enable comparable contribution of each gene member to the module score, followed by mean value calculation across the transformed genes in the module for each sample.

To assess cellular contributions for each pathway, the non-adjusted expression of each gene in the dynamic pathways was regressed over the major peripheral cell type frequencies as determined by CyTOF including granulocytes, CD4 and CD8 T cells, B cells, NK cells and monocytes. The cell-specific contribution to each pathway was determined as the mean of the coefficients of the tested cell type across all genes in the module.

The centrality of each pathway in the response network was also evaluated by calculating the pathway based mean betweenness across all gene members of the pathway. To further assess the clinical relevance of the dynamic pathways to the treatment response, the calculated pathway score at all tested time points was correlated with CRP using Spearman's rank correlation test.

### ‘Disruption-Network’ Analysis as a Framework for Mapping Person-Specific Non-Response Profile to a Global Response Network

To understand individual variation in non-response dynamics, the inventors developed an approach termed ‘disruption network’ in which individual non-responders are iteratively added to the obtained normal anti-TNF response network, and the disruption in the correlation structures is assessed for each edge in the reference response network. The disruption is evaluated in the node (gene/cell) or the module level to determine biological mechanisms that may explain patterns of the non-response.

More specifically, consider a feature matrix F n×m where n is the number of samples for a given condition, in our case, n is the number of samples of responding patients and m is the number of features, where f(i,j) refers to a fold change measured value at a given time point relative to baseline, of the j-th feature in the i-th sample. Let matrix R m×m be the feature pairwise Spearman's rank correlation matrix based on F which represents the global response network, where r(j,k)=cor(j,k) for genes j and k. Insignificant correlation values according to FDR thresholds, as described above, were presented as NAs in the matrix.

The ‘disruption network’ construction was assessed individually for each non-responder as follows: a new F′(n+1)×m matrix was generated by the addition of the tested non-responder to the responders' samples. Based on F′, a new pairwise Spearman's rank correlation matrix was calculated to obtain R′ m×m, in which r′(j,k) is the correlation between j and k genes when including the non-responder in the responders' samples.

For correlation coefficients comparison, correlation coefficient values were transformed using Fisher z-transformation by the following formula:

$$z ( r ) = 0.5 * ln ( \frac{1 + r}{1 - r} )$$

and a standard error of

$$SEz ( r ) = \frac{1}{\sqrt{n - 3}}$$

where n is the number of samples. The inventors define a ‘disruption’ term as the drop in the Fisher z transformed values between two genes as a result of the non-responder addition using the statistical z score which is defined as:

$$disruption ( j , k ) = z score = \frac{z ( r ′ ) - Z ( r )}{Pooled \cdot SEz} = \frac{z ( r ′ ) - Z ( r )}{\sqrt{\frac{1}{( n + 1 ) - 3} + \frac{1}{n - 3}}} .$$

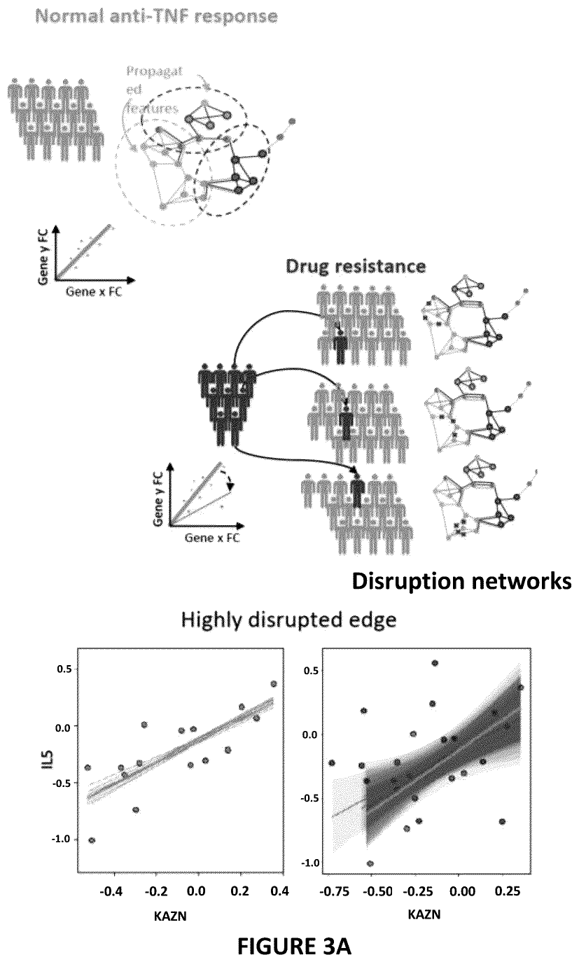

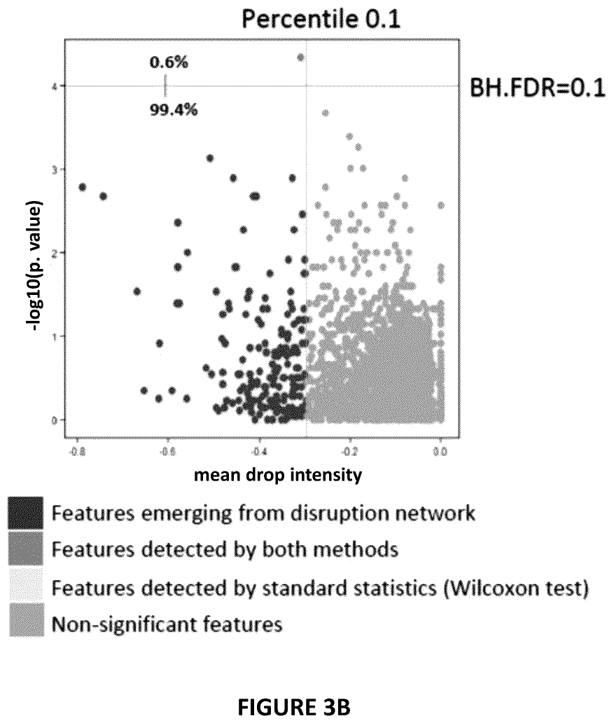

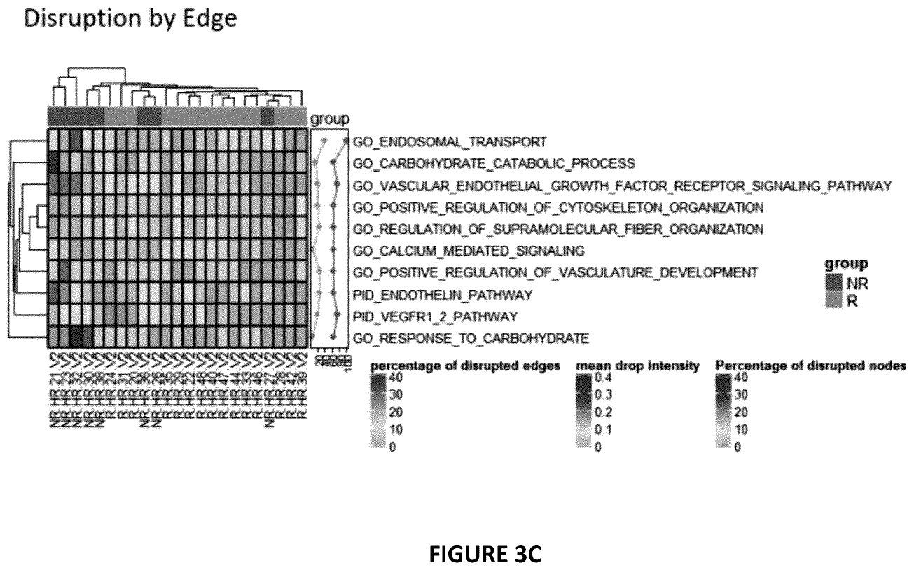

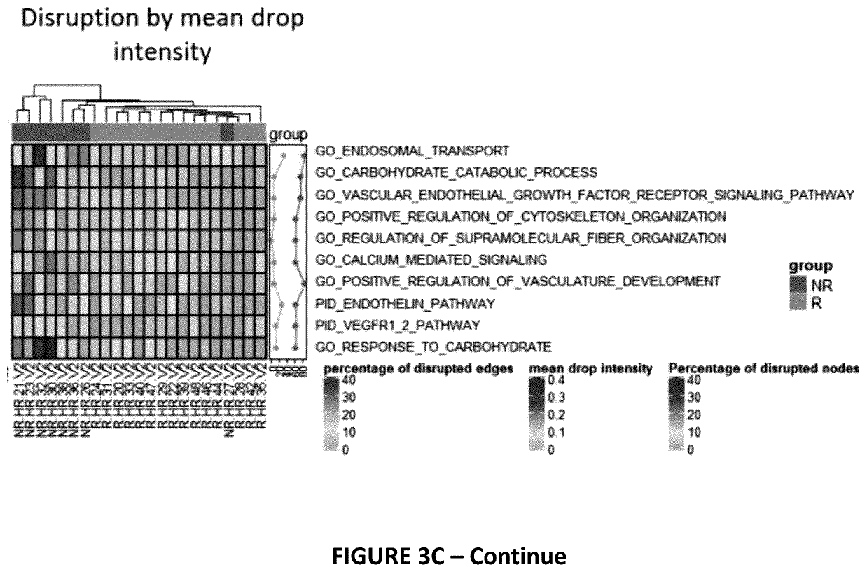

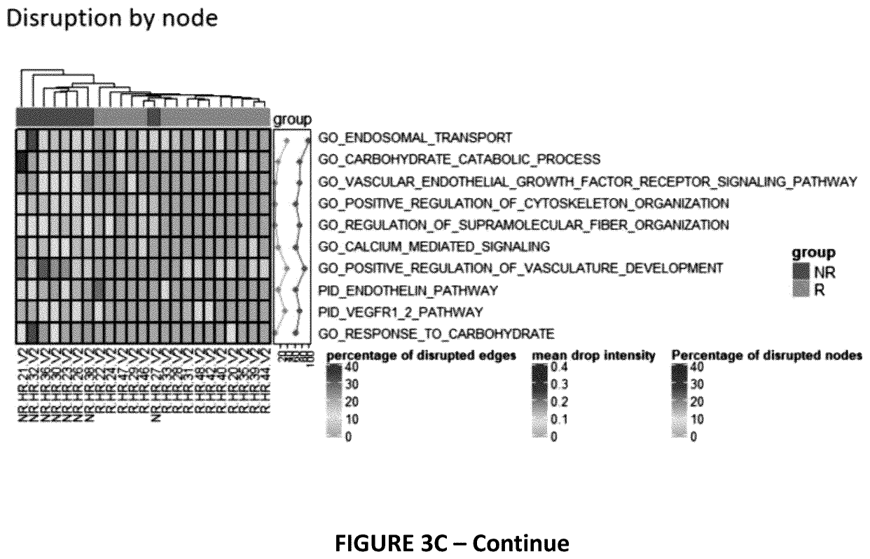

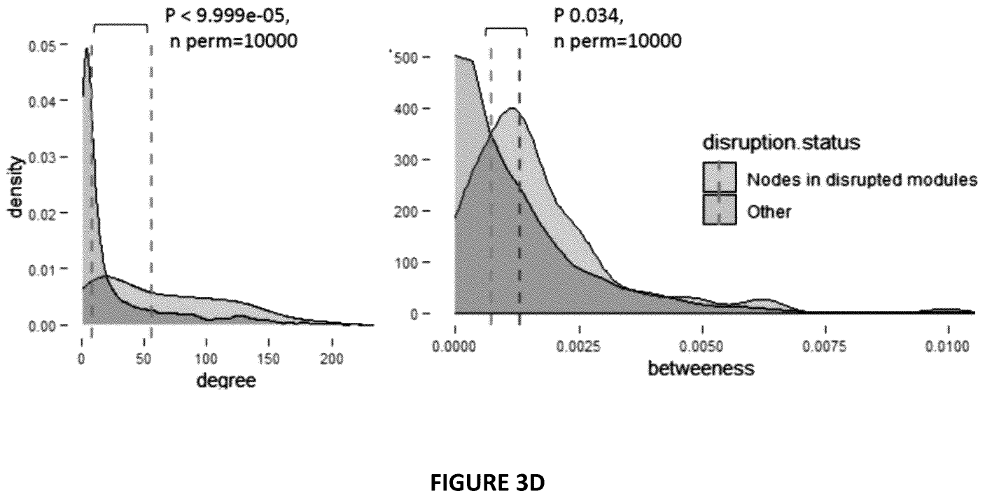

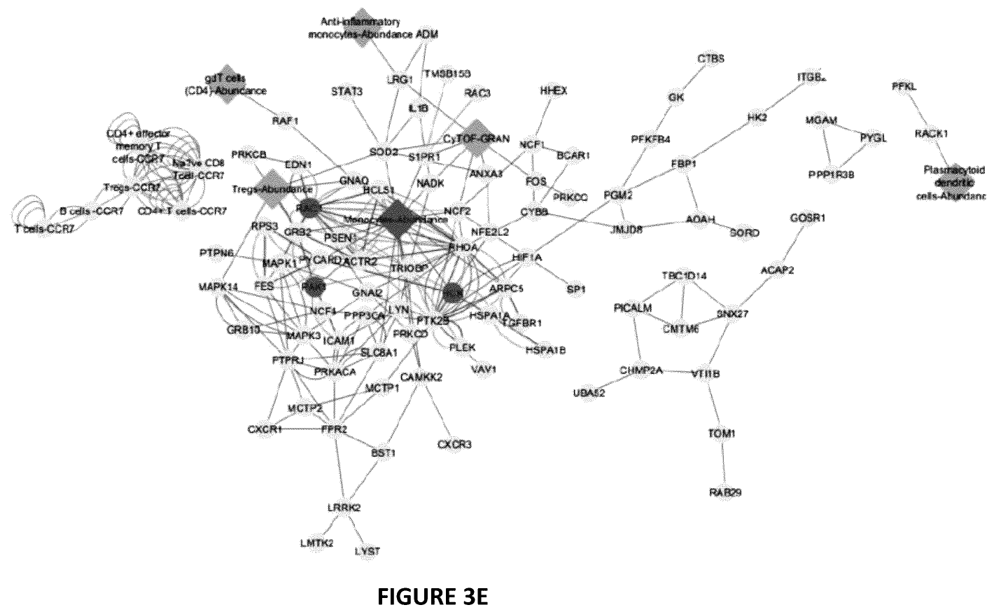

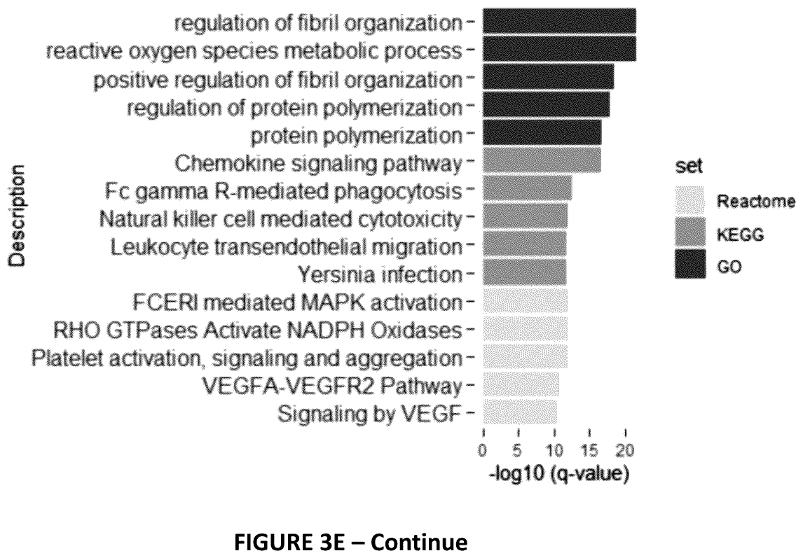

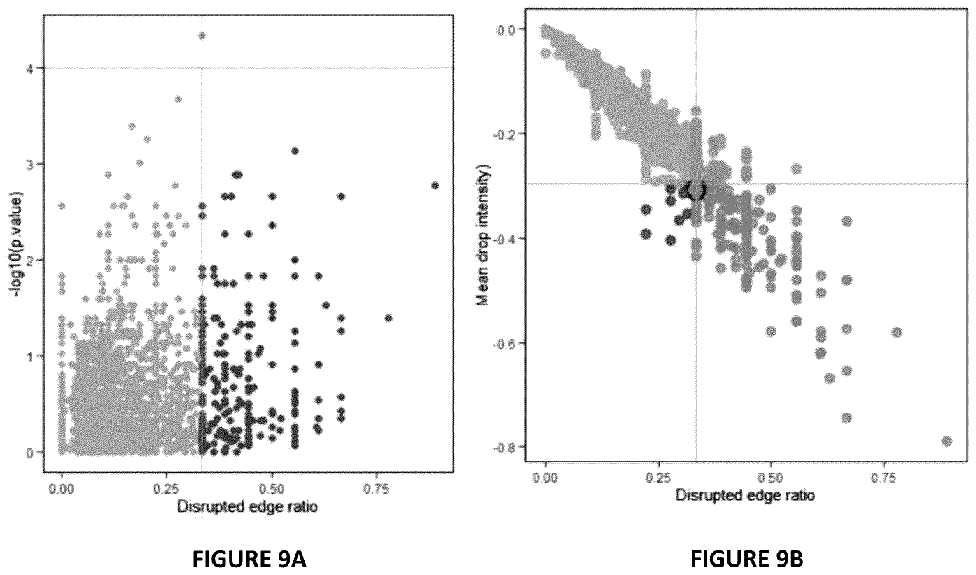

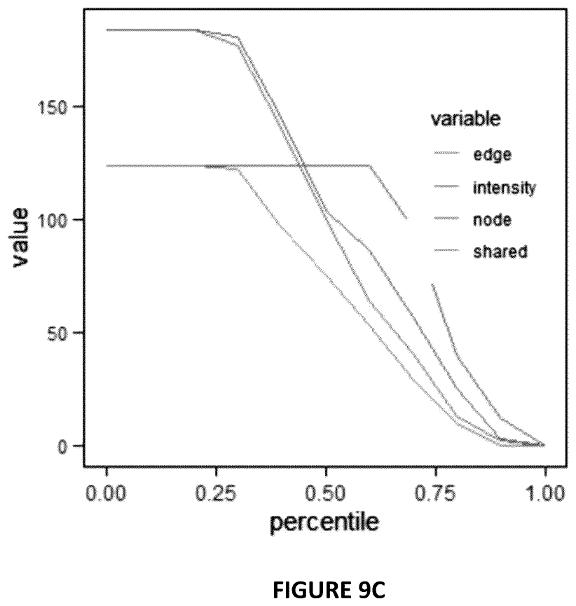

Only negative values of sign(r*(z(r′)−Z(r))), which indicate weakening of the original correlation obtained in responders were included, while positive values were set to zeros. Drop degree of confidence for non-responders was assessed empirically for each drop value in each edge, based on the non-responder drop value percentile in the responders' normal drop distribution. This was further corrected for multiple testing using the Benjamini-Hochberg procedure. Edges with drop adjusted percentile<0.1 were considered as significantly disrupted. Insignificant drop values were set to zeros. Analysis of disruption parameters in the feature level, revealed a considerably expansion of the detected differential signal between response groups, compared to standard differential analysis by Wilcoxon test (biorxiv.org/content/10.1101/2021.06.16.448558v1.supplementary-material?versioned=true). While using the Wilcoxon test, the inventors detected only one feature (0.06%), with significant differential dynamics between response groups at W2, the inventors identified this feature together with 179 additional features (10%) when using disruption parameter of top mean drop intensity (FDR<0.1 by Wilcoxon test, FDR<0.1 for significant dropout and top 0.1 percentile of mean drop intensity, FIG. 3B). The inventors observed similar results for the disrupted edge ratio (0.06% Vs. 14.4% significant features identified by Wilcoxon test (FDR<0.1) and top disrupted edge ratio parameter (FDR<0.1 for significant dropout and top 0.1th percentile of node disrupted edges) respectively, FIG. 9A). Testing the agreement of both disruption parameters, the inventors identified 9.4% dynamics differential features including the single feature identified by Wilcoxon test (FIG. 9B).

Disruption was also measured in the pathway level for each individual using three different measurements: (1) Module specific mean drop intensity in which a mean drop intensity was calculated across the relevant edges in the module, for a specific individual. (2) Module specific percentage of disrupted edges which determines the percentage of edges in the module that the specific individual is significantly disrupted in. (3) Module specific percentage of disrupted nodes which evaluate the percentage of disrupted nodes for a specific individual out of all module nodes.

For binary classification of disrupted pathways, the inventors quantify the disruption measure across a range of percentile values in each parameter. For each parameter, in each percentile, the selected positive disrupted modules were those that were disrupted in at least 50% of the non-responding patients and in less than 20% of the responders, or in cases where the difference between the percentage of disrupted non-responders to responders is higher than 50%. The top significantly positive disrupted modules were defined as those with a complete agreement of all three parameters in the highest percentile with shared selected pathways across all parameters, which in our case was determined as the 0.8 percentile.

### Peripheral Blood Mononuclear Cells (PBMCs) Cryopreservation

Blood samples were drawn before Infliximab first infusion. PBMCs were isolated using density gradient centrifugation by spinning blood over UNI-SEPmaxi+ tubes (Novamed Ltd.) following the manufacturer's protocol. Isolated cells were resuspended in 1 mL freezing solution, containing 10% DMSO and 90% FCS. The samples were kept in Nalgene Mr. Frost® Cryo 1° C. Freezing Container (ThermoFisher scientific) with Isopropyl alcohol at −80° C. over-night, and immediately after placed in a liquid nitrogen container for long-term storage.

### Single Cell RNA Sequencing in 10× Genomics Platform

PBMCs from responder and non-responder patients pre-treatment (N=2) were prepared for scRNA-seq according to the 10× Genomics Single Cell protocols for fresh frozen human peripheral blood mononuclear cells. Briefly, Frozen PBMCs were rapidly thawed at 37° C. and transferred into 50 mL centrifuge tubes. The cryovials were rinsed with 1 mL of warm (37° C.) RPMI 1640 supplemented with 10% of FCS which was added dropwise to the DMSO containing fraction while gently shaking the cells. Next, the cells were sequentially diluted by first adding 2 mL of medium followed by another 4, 8 and 16 mL, respectively, with 1 min wait between the four dilution steps. The diluted cell suspension was centrifuged for 5 min at 300 g. Most of the supernatant was discarded leaving ˜1 ml, and the cells were resuspended in 9 ml of medium followed by additional centrifugation for 5 min at 300 g. The cells were adjusted to a final cell concentration of 1,000 cells/U1 and placed on ice until loading into the 10× Genomics Chromium system. The scRNA sequencing was performed in the genomic center of the biomedical core facility in the Rappaport faculty of medicine at the Technion-Israel Institute of Technology. Libraries were prepared using 10× Genomics Library Kits (Chromium Next GEM Single Cell 3′ Library & Gel Bead Kit v3.1, PN-1000121) using 20,000 input cells per sample. Single cell separation was performed using the Chromium Next GEM Chip G Single Cell Kit (PN-1000120). The RNAseq data was generated on Illumina NextSeq500, high-output mode (Illumina, FC-404-2005), 75 bp paired-end reads (Read1—28 bp, Read2—56 bp, Index—8 bp).

### Single Cell Data Analysis

Cell Ranger single cell software suite was used for sample de-multiplexing, alignment to human reference genome (GRCh38-3.0.0), cell barcode processing and single cell UMI counting following default settings. The UMI count matrix was further processed using the Seurat R package (version 3.1.4). First, as a QC step, cells that had a unique feature count of less than 200 were filtered out. Additional filtering was applied to remove features detected in less than 3 cells. The inventors further filtered cells based on mitochondrial gene content above 0.25%. After this step, 19,275 single cells and 20,673 genes in total were retained and included in downstream analyses. This was followed by Global-scaling library size normalization. Genes were scaled in comparison to all other cells and regressed out the effects of unwanted sources of variation including UMI counts and percentage of mitochondrial genes for the remaining cells. At the next step, the inventors performed linear dimensionality reduction on the scaled data of the top 2,000 highly variable genes. Resampling test based on the jackstraw procedure and Elbow plot were performed to identify the first 30 significance principal components that were used for downstream visualization by t-SNE plot.

SingleR 77 was used to annotate cell types based on correlation profiles with two different resolutions of cell classification using the Blueprint-Encode 78 and the Monaco Immune Cell (GSE107011 79;) reference datasets of pure cell types. Differential expression analysis between responders and non-responders was performed for each cell population using a Wilcoxon Rank Sum test implemented in the FindAllMarkers function in the Seurat package.

Coexpression network for the pre-treatment differential RAC1-PAK1 core differential seed, including HCK, RAC1, PAK1, HCK, GRB2, ICAM1, and EDN1 was constructed for each monocyte subset in each response group. The networks were expanded using 3 additional iterations, where in each iteration the core seed genes used contained the network nodes obtained in the previous iteration. Global functional enrichment was calculated by a hypergeometric test based on the Reactom database using the Clusterprofiler R package. Wilcoxon test was assessed to identify significant differences in module scores between response groups for each enriched pathway in each monocyte subset. p values were further adjusted for multiple testing using the Benjamini-Hochberg procedure.

### Example 1

### Treatment Response Pushes Individuals Along an Immune Health Axis, Whereas Non-Responders Shift Back

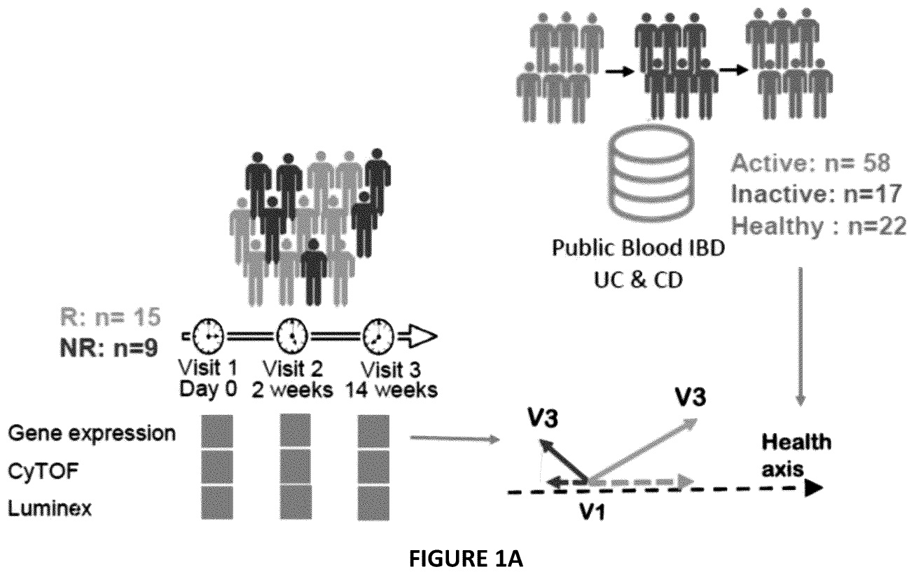

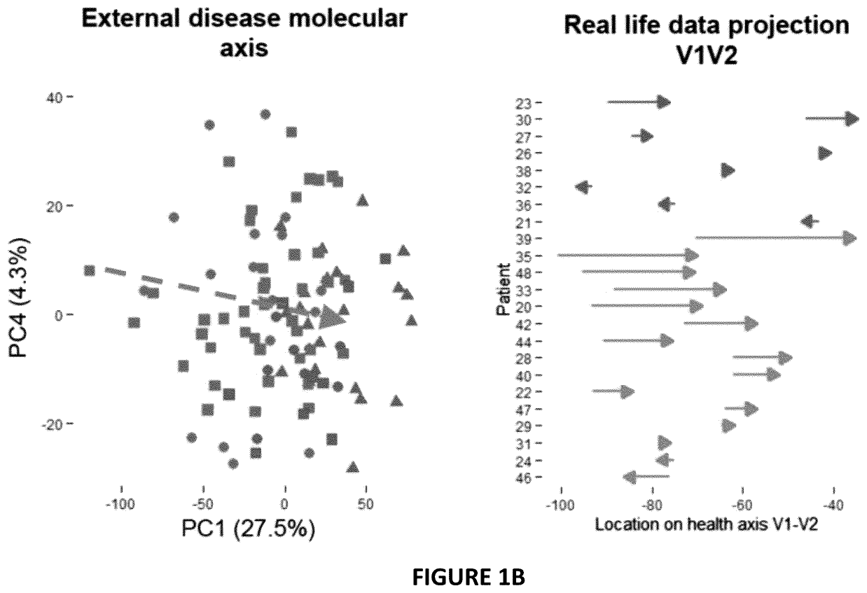

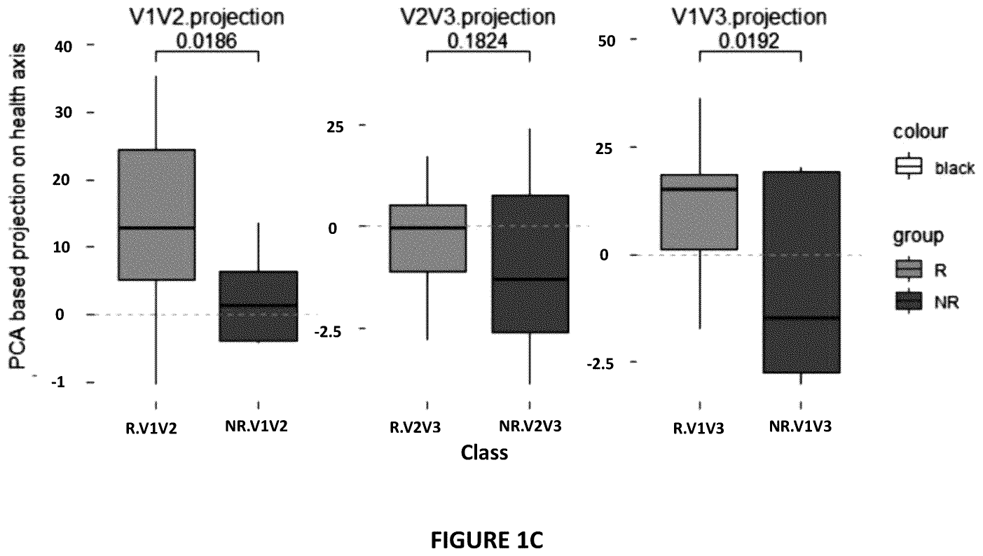

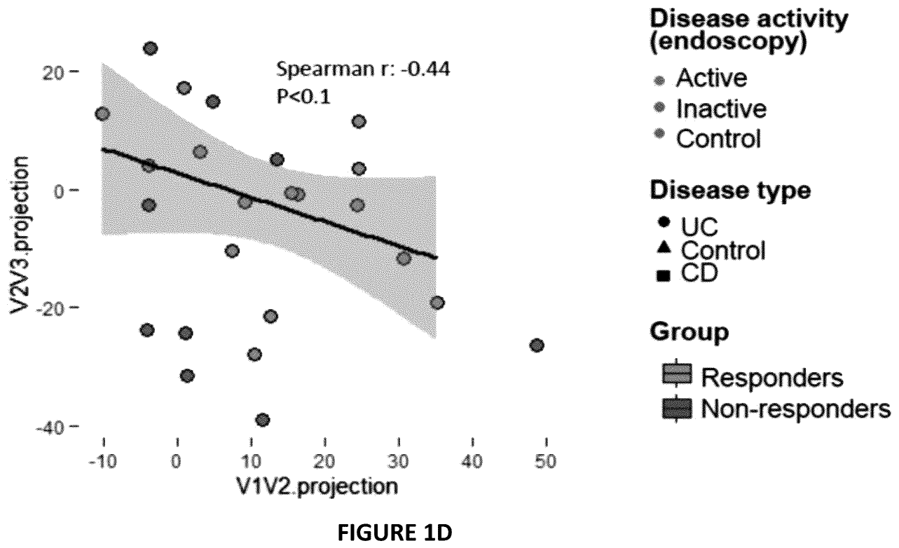

To understand the cellular and molecular changes associated with anti-TNFα response and resistance, the inventors performed longitudinal deep immunophenotyping of peripheral blood in Crohn's disease (CD) patients who received first-time therapy with Infliximab during IBD standard clinical care (FIG. 1A, left, hereon infliximab cohort). Patients were profiled a total of three times, once pre-treatment, with additional time points collected at week 2 and week 14 post-treatment initiation. At week 14, a total of 15 patients showed clinical response to Infliximab (IFX) whereas 9 patients were classified as non-responders at the end of the study follow-up period (biorxiv.org/content/10.1101/2021.06.16.448558v1.supplementary-material?versioned=true; see Methods for response classification).

Complementary to this, to define an individual-specific unbiased expectation of changes in blood during IBD and how they relate to health, the inventors used a public gene expression dataset of whole blood samples from healthy individuals and 75 IBD patients (25 UC, 50 CD) in varying disease states and treated with standard of care drugs (FIG. 1A, right; see Methods). The inventors constructed an external data-driven reference IBD axis (FIG. 1B, Left) which describes in a dimensionality reduced PCA (Principal Component Analysis) space the molecular transition from active- through inactive disease to healthy-state, based on differentially expressed genes in blood (hereon ‘Health axis’, see Methods). Next, the inventors projected the position of an in-house Infliximab cohort on the PCA (FIG. 1B, Right) and calculated for each patient the distance they traversed on the axis, providing continuous molecular information to characterize a patient's immune disease state shift (FIG. 1C). Analyzing the distance between paired sample time points, the inventors observed that responders progressed on the health axis (e.g., a positive shift on the axis towards the centroid of healthy reference samples) while non-responders regressed on it (FIG. 1C, p<0.05, one sided permutation test). Breaking up these dynamics by time point, the inventors observed that responders exhibited increased progress along the health axis following first drug treatment, and reduced progress after the second treatment (FIG. 1C). The negative correlation between progress along the axis between V1-V2 and progress in the following segment V2-V3 suggest that patients progressing to ‘response’ early, slow down during the following visit whereas those showing a slow progress initially progress more thereafter (FIG. 1D). The herein disclosed results suggest that non-responders are affected by treatment as well, demonstrating immune dynamics following IFX treatment (by treatment or time) with an overall opposite direction from responders' progress. Collectively, the herein disclosed health axis, captures in blood molecular changes which are clinically relevant for treatment response.

### Example 2

### Early IFX Normal Response Reduces Expression of Innate Immune Related Pathways Attributed Mainly to Monocytes Function

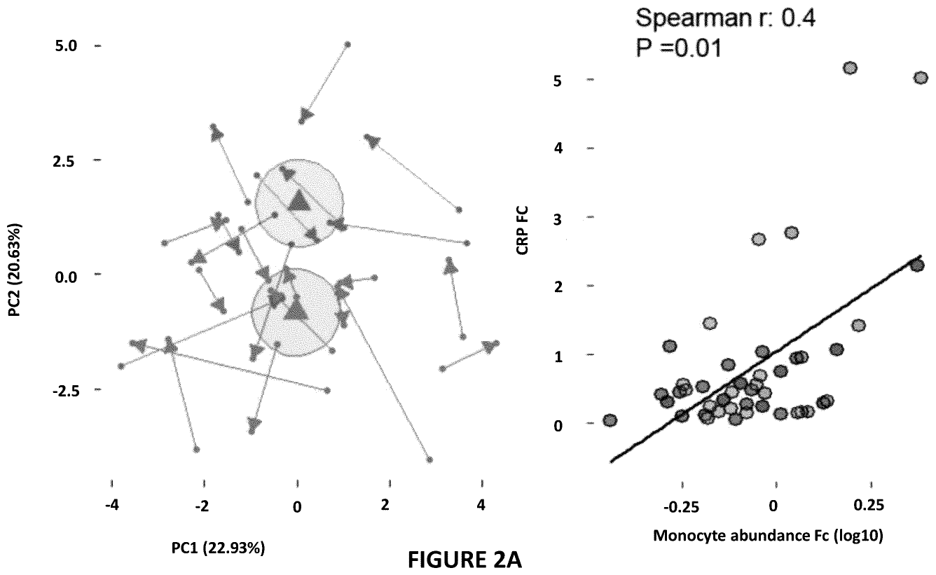

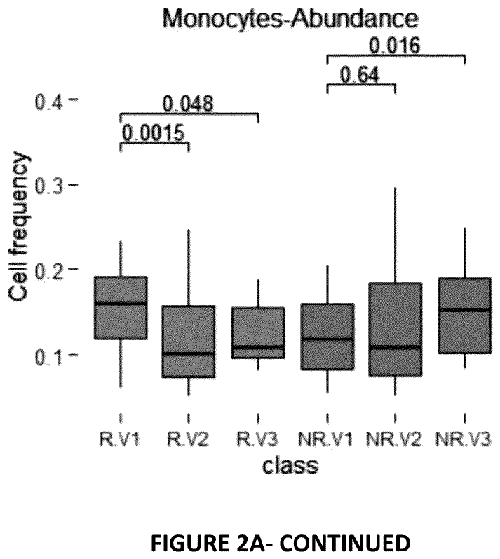

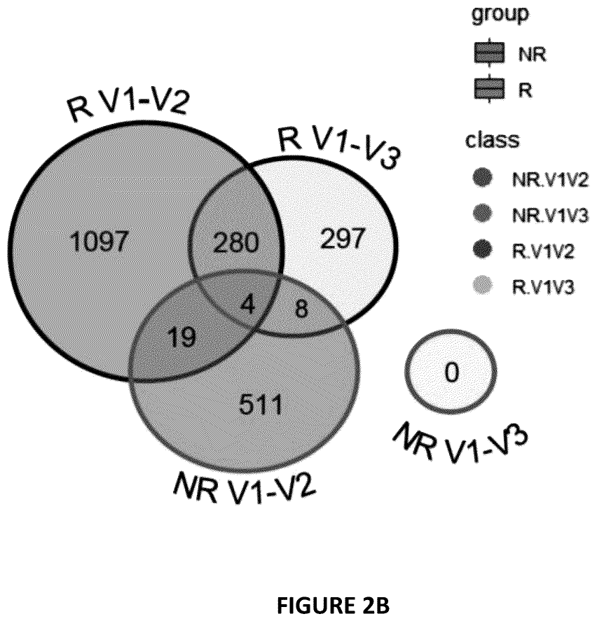

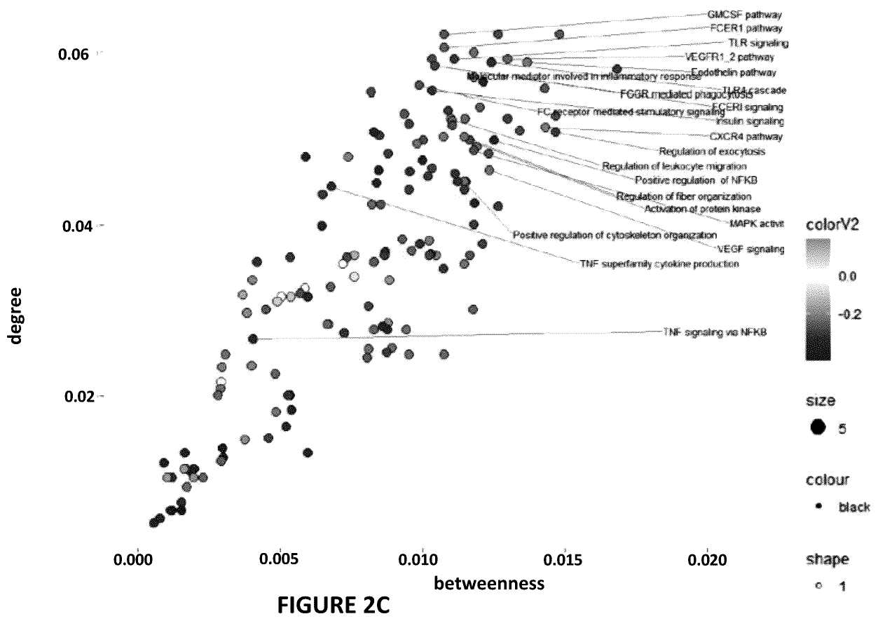

To identify changes following treatment in each response group, the inventors characterized major immune cell compositional changes into 16 canonical immune populations (FIG. 2, and biorxiv.org/content/10.1101/2021.06.16.448558v1.supplementary-material?versioned=true). Then, to compare how peripheral blood state differs as a function of treatment response, the inventors computed a PCA on the fold change patients' cell phenotyping profiles (FIG. 2A, left). The inventors observed significant difference in cell abundance changes between responders and non-responders for W2 and W14 changes relative to baseline (P=0.005, NPMANOVA).

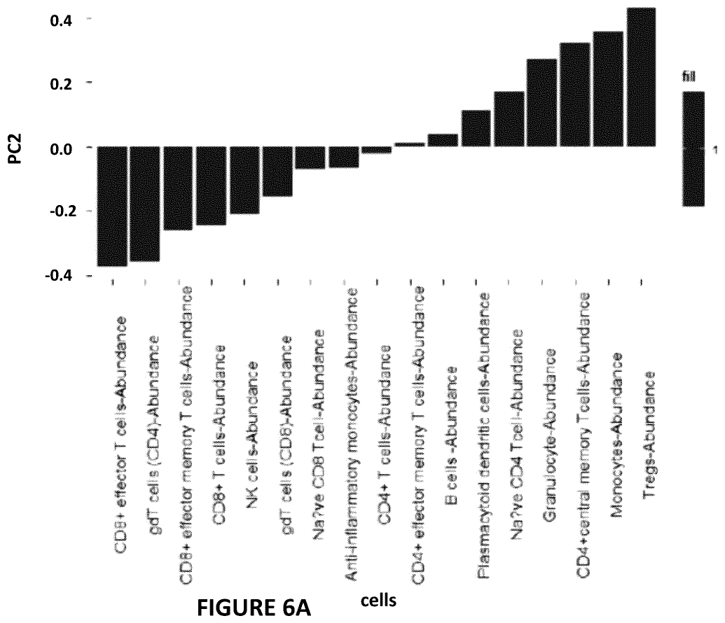

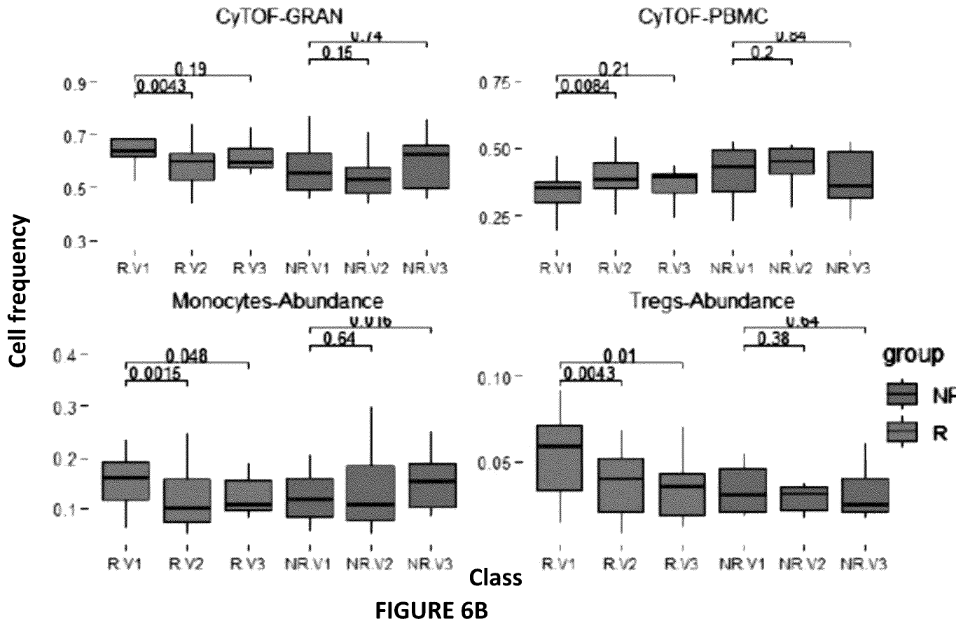

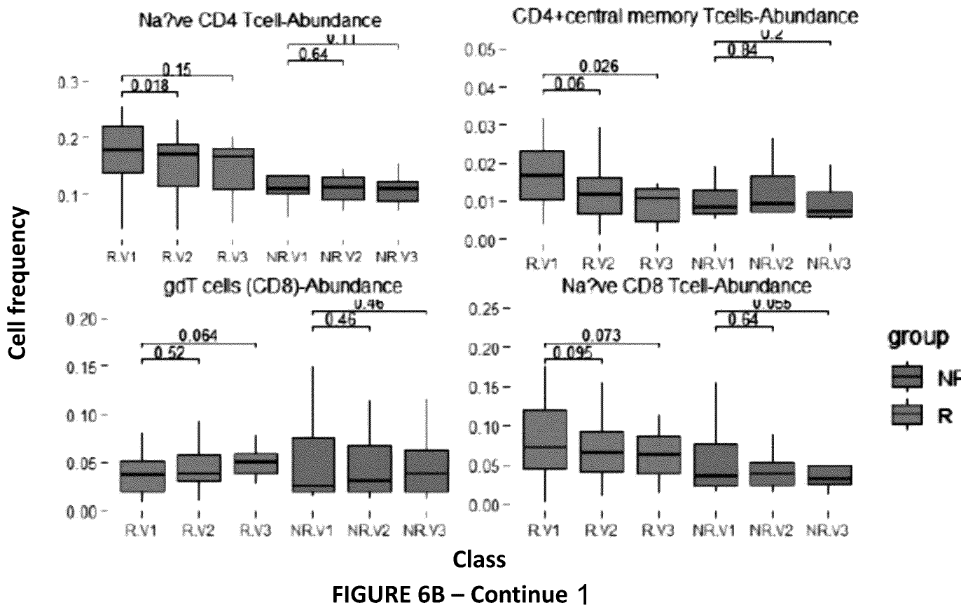

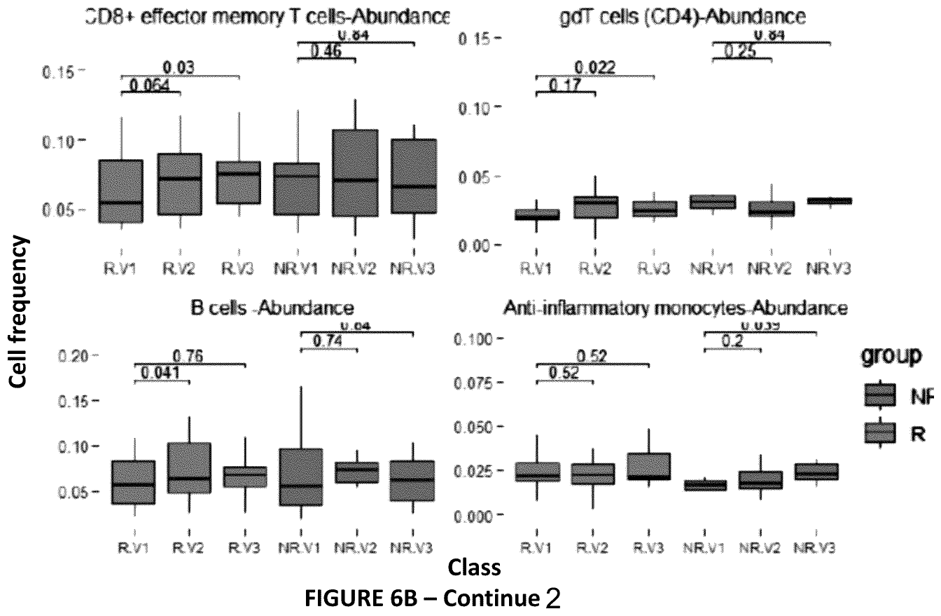

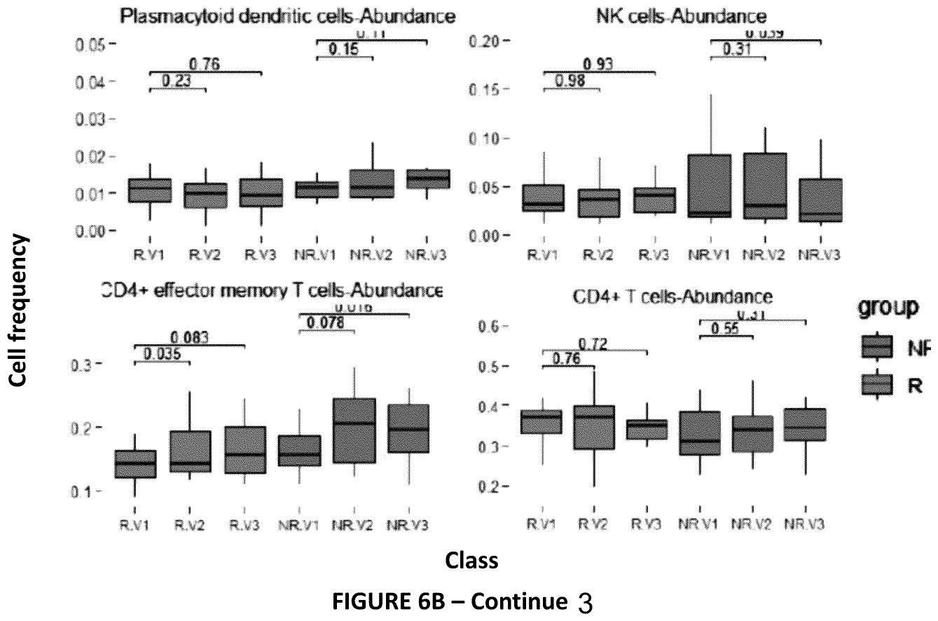

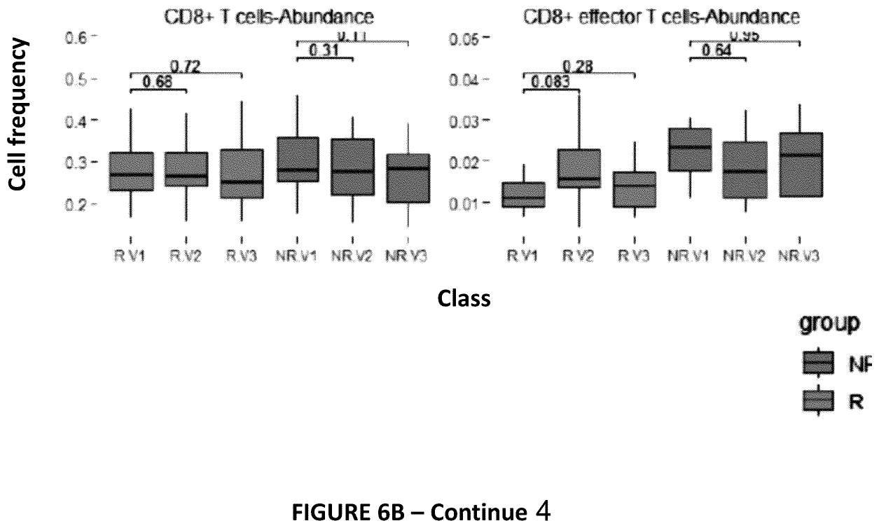

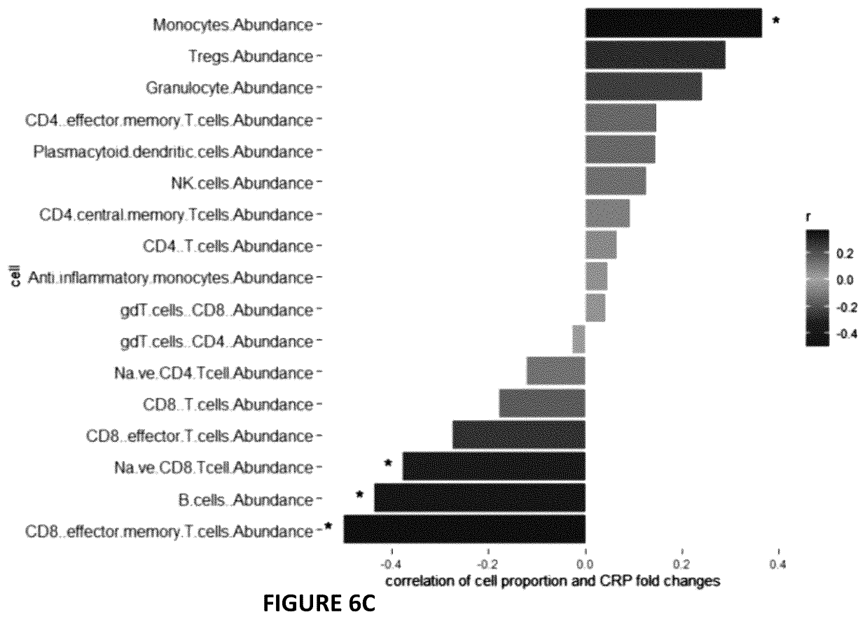

Multiple cell subset changes in responders were apparent already in the early W2 response period including reduced abundance of monocytes, granulocytes, Tregs, naïve CD4+ T cells, CD4+ central memory T cells and increased abundance of CD4+ and CD8+ effector memory T cells and B cells (FDR≤0.15, Paired Wilcoxon test; FIG. 6B). Based on the PCA loadings the inventors deduced that monocytes and Tregs where the prime drivers of changes following treatment, evidence for which was also supported by the univariate comparison showing that monocytes were significantly reduced in responders throughout all time points, whereas in non-responders monocytes' frequency was unchanged in W2 and in the reverse direction at W14 (p=0.0015 and p=0.048 for reduced frequency in responders, as opposed to P=0.64 and P=0.016 in non-responders, at W2 and W14 respectively, Paired Wilcoxon test). Moreover, monocyte frequency was also correlated with changes in CRP (Spearman's r=0.4, p=0.01), suggesting their relevance to treatment response (FIG. 2A center, right and FIG. 6C for correlation of CRP with other cell-types). Taken together, our results demonstrate significant differential cell composition following IFX treatment as a function of response, with monocytes likely playing a major role.

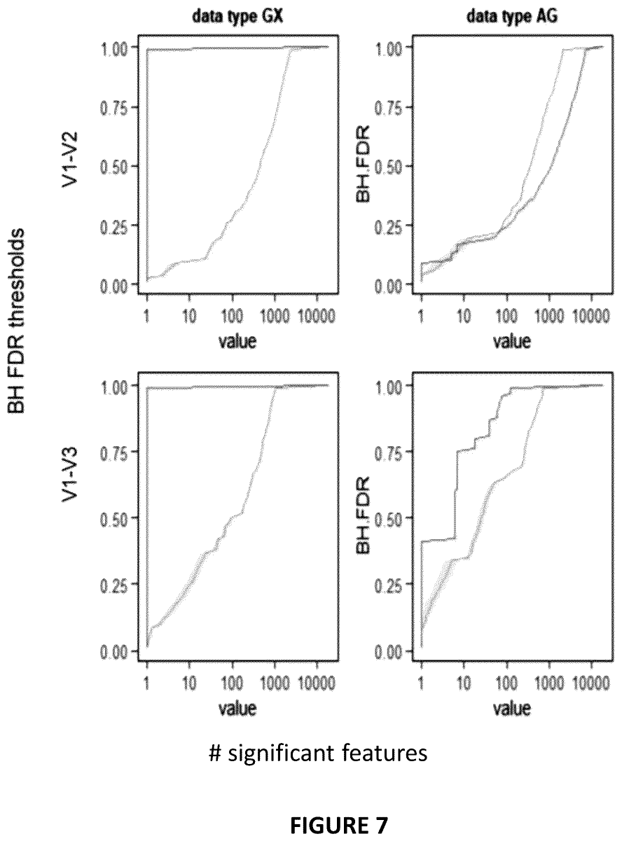

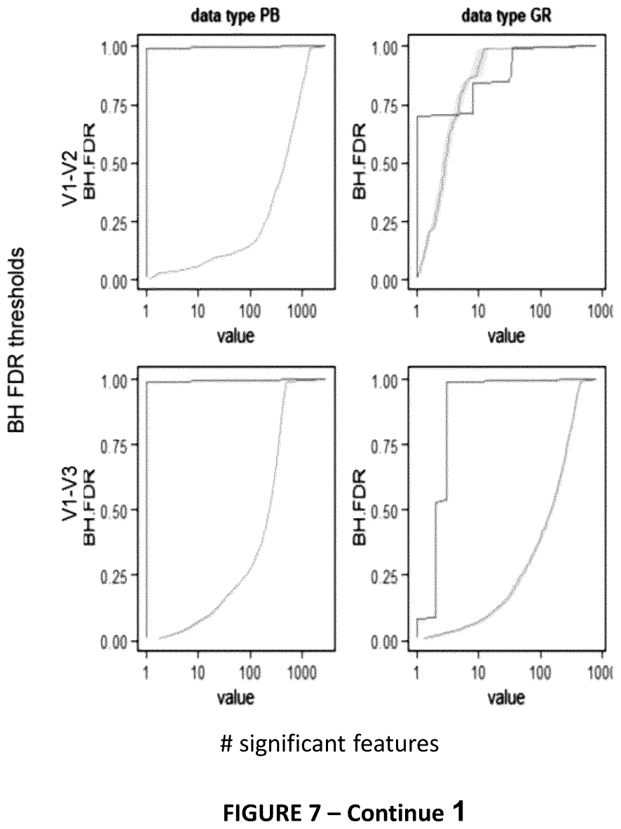

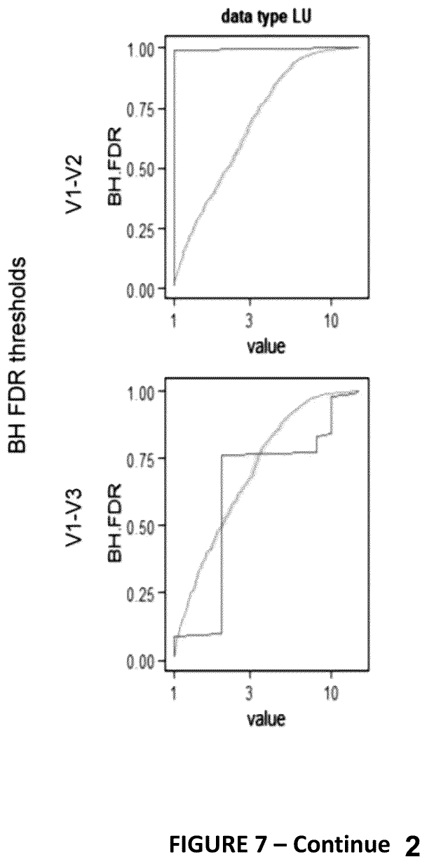

Given the observed cell composition alternations, the inventors performed a cell-centered analysis to identify changes in transcriptional programs following treatment in each response group, by adjusting the gene expression for variation in major cell type proportions. This procedure places focus on detection of differences between conditions of the gene regulatory programs the cells are undergoing rather than those differences due to underlying cell compositional differences and has been shown to unmask additional signal (e.g., false-negative of direct bulk analysis) while decreasing false-positives (FIG. 2B, see Methods; Gaujoux et al., 2019). In this analysis, the inventors identified 1,400 (5.99%) and 589 (2.52%) differential features in responders (FDR<0.15, permutation test; and biorxiv.org/content/10.1101/2021.06.16.448558v1 .supplementary-material?versioned=true) at W2 and W14 compared to baseline respectively, suggesting enhanced response at W2 followed by reduced dynamics in W14. Compared to responders, non-responders showed attenuated dynamics in the parallel treatment periods, with only 542 (2.32%, biorxiv.org/content/10.1101/2021.06.16.448558v1.supplementary-material?versioned=true) differential features at W2 compared to baseline, and no significantly detected dynamics at W14. To ensure the differences in dynamics between the two response groups was not due to sample size, the inventors subsampled responders to match the non-responder group size and observed that responding individuals harbor more dynamic changes than those classified as non-responders (FIG. 7). Furthermore, comparing the two response groups the inventors observed only a minor overlap in the features whose dynamics change post-treatment (23 features, 1.2% at W2). In line with the ‘health axis’, these results suggest that there is increased early dynamics in responders compared to non-responders and that responders and non-responders presented different alternations following treatment.

To understand the interactions between the altered features during normal IFX response and to put them into biological context, the inventors constructed a cell-centered co-expression network, which was expanded by known interacting genes, followed by functional enrichment analysis (see Methods and biorxiv.org/content/10. 1101/2021.06.16.448558v1 .supplementary-material?versioned=true). Interestingly, despite this being a blood-based network, the inventors noted genes which were previously associated with anti-TNF response in IBD biopsy such as TREM1 and OSM, suggesting that recovery of relevant signals originally detected in tissue, are also reflected in blood. The inventors identify potential putative mediating pathways, possessing higher connectivity to other nodes in the response network, according to their averaged degree and betweenness centrality measurements (FIG. 2C).

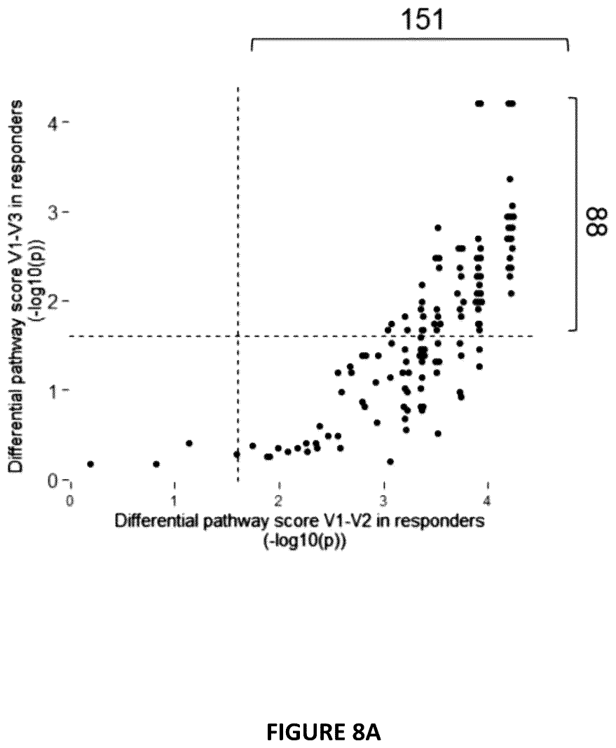

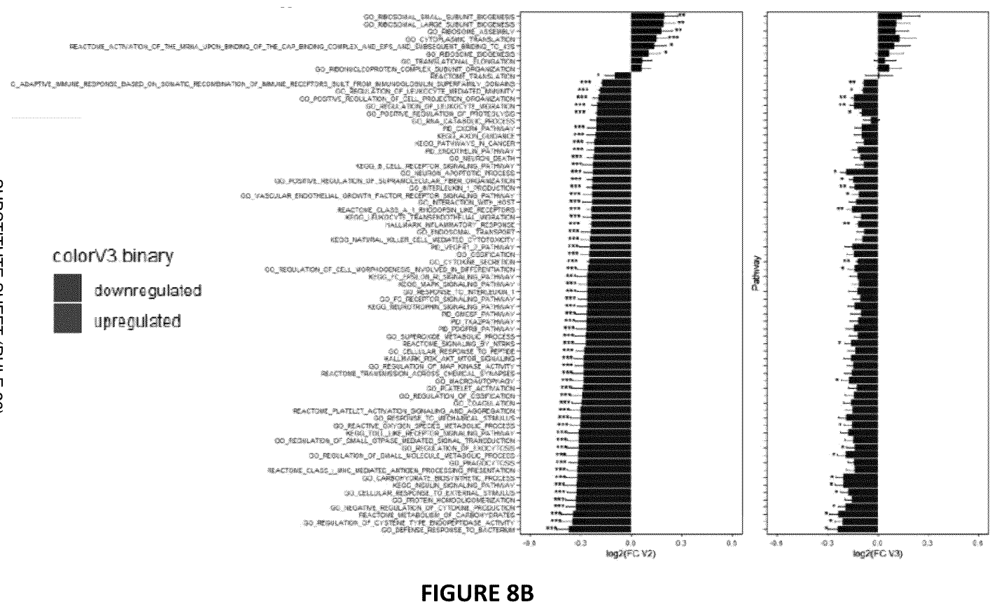

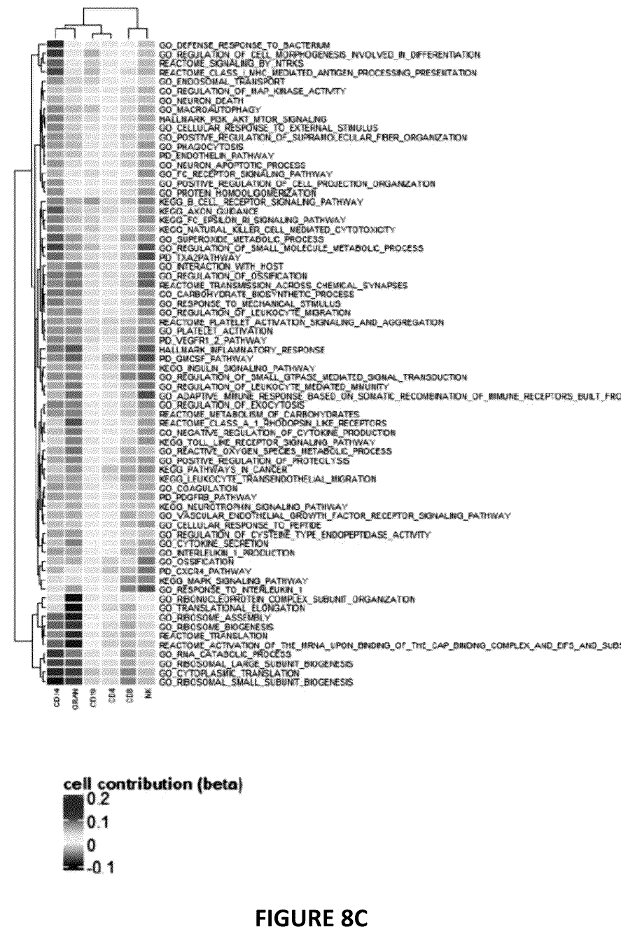

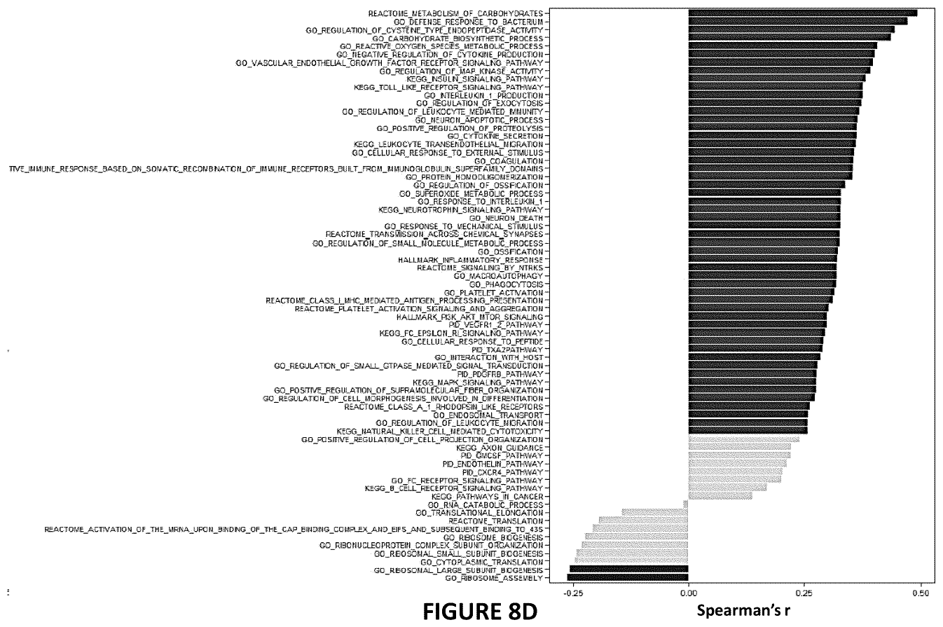

The inventors observed that most central pathways associated with the 2W early response were related to the innate immune system (FIG. 8B and biorxiv.org/content/10.1101/2021.06.16.448558v1 .supplementary-material?versioned=true). At the pathway level, consistent with the ‘health axis’ and feature level analysis, the inventors found augmented response at W2, which was attenuated in the later response stage (151 vs. 88 enriched dynamic pathways in responders at W2 and W14, respectively; FIGS. 8A-8B). As expected, among the innate related altered functions following efficient treatment response, the inventors observed pathways related to downregulation of NF-kB and TNF signaling via NF-KB (FIG. 2C, FDR<0.005 for W2 vs. baseline, by Wilcoxon test; FDR<0.01 for enrichment by fGSEA,). Pathways with high network centrality included downregulation of FC receptor signaling and phagocytosis, cytoskeleton organization, Toll-like receptors (TLRs) and vascular endothelial growth factor (VEGF) signaling responses (FIG. 2C; >75th percentile for both degree and betweenness; FDR<0.005 for W2 vs. baseline, by Wilcoxon test; FDR<0.1 for enrichment by fGSEA), which were also correlated with clinical CRP (Spearman's r FDR<0.05 and FIG. 8D). Of note, FCYR is known to be regulated by TNFα25 and mediates a number of responses, including the phagocytosis of IgG-coated particles, accompanied by cytoskeleton rearrangements and phagosome formation, central pathways that were downregulated in responders (FIG. 2C and FIG. 8B, FDR<0.001 for W2 vs. baseline, by Wilcoxon test; FDR<0.15 for enrichment by fGSEA). The inventors also observed the downregulation of reactive oxygen species pathway, downstream to FCYR receptor signaling, which is crucial for the digestion of engulfed materials in phagosomes (FDR<0.001 for W2 vs. baseline, by Wilcoxon test; FDR<0.05 for enrichment by fGSEA). This pathway also presented significant correlation with CRP (Spearman's r 0.43, FDR<0.005, FIG. 7B and FIG. 7D). To identify the cell most likely expressing these pathways, the inventors regressed the unadjusted fold change gene expression on major blood immune cell abundance changes (see Methods). The inventors observed that monocytes and granulocytes were the major contributors associated with the dynamic pathways (FIG. 8C). This further supports the considerable contribution of monocytes to the normal treatment response, on top of their significant frequency alternation and their frequency correlation with CRP.

### Example 3

### Disruption Networks' as a Framework to Understand Individual Variation in Non-Responders' Dynamics

Whether non-responders' transcriptional profile reflects fundamental routes of IFX drug resistance, is essential for tailoring treatment. To elucidate molecular mechanisms of individual-specific pathways of non-response to treatment, the inventors devised a systematic framework termed herein ‘disruption networks’, whose underlying principle is the study of relations between features and their inference of how these relations differ at the individual sample level, providing inference of how each individual's molecular network behaves in a specific condition.

To identify how non-responding individuals differ with respect to the normal IFX response dynamics, the inventors iteratively added a single individual non-responding patient to the obtained normal response reference network and calculated the disruption in the correlation structure in each edge for that patient (hereon ‘dropout’). This procedure was performed separately for each non-responder. The inventors considered only negative dropouts, that is, events in which the relation (e.g., correlation) between two features was weakened once the non-responder was spiked into the responders' group, indicating deviance from normal treatment response (FIG. 3A right, for an example). To evaluate non-responders' dropout significance, the inventors generated empirical null distribution of dropouts (‘normal response’ dropouts) by iterative addition of each responder's sample to the other responders' samples. The inventors calculated P-values as a left tail percentile, within the null distribution of the normal dropouts, which were further corrected for multiple testing (FIG. 3A; see Methods). By applying the disruption networks framework, the inventors considerably expanded the detected differential signal between response groups, compared to standard differential analysis (1 feature (0.06%) by Wilcoxon test (FDR<0.1) vs. 180 features (10%) by top mean drop intensity, including the single feature identified by Wilcoxon test (FDR<0.1 for dropout significance and top 10^{th} percentile of mean drop intensity); FIG. 3B and FIGS. 9A-9B for mean drop intensity, disrupted edge ratio parameters and the agreement of both respectively).

To understand disruption in the functional context, the inventors aggregated the dropouts to calculate a pathway-level personalized disruption, according to three parameters including percentage of disrupted edges, percentage of disrupted nodes and mean drop intensity per gene-set for each individual and identified top disrupted pathways by full agreement across all parameters (FIG. 3C and biorxiv.org/content/10.1101/2021.06.16.448558v1.supplementary-material?versioned=true; see Methods). The inventors found that the major disrupted regulatory programs at W2 were related to the cytoskeleton/fiber organization and VEGFR signaling, which were central functions that showed significant downregulation also during normal treatment dynamics in responders. Interestingly, by comparing node centrality of the major disrupted pathways to other nodes in the response network, the found that the highly disrupted nodes had significantly higher centrality in the overall response network as measured by betweenness and degree values (P<9.999e-05 and p=0.034 for degree and betweenness correspondingly by permutation test, n perm=10,000; FIG. 3D). On the meta-pathway level (biorxiv.org/content/10.1101/2021.06.16.448558v1.supplementary-material?versioned=true), the inventors observed that monocytes are highly associated with the disrupted pathways, presenting high centrality (degree: at the 96.7^{th} percentile; betweenness: at the 100^{th} percentile) in the disrupted pathways' sub-graph (FIG. 3E, left). The disrupted pathways share cellular events that couple multi-subunit immune-recognition receptors (MIRRs) to their various effector functions. These pathways include the core genes of the HCK-RAC1-PAK1 signaling cascade with significantly high combined centrality of degree and betweenness (p=0.017, n=1,000 random triple node subsampling). The core perturbed axis is a final common pathway involving intracellular signaling through several proximal receptor tyrosine kinases and co-receptors, which induces phosphorylation and activation of SFKs (SRC family tyrosine kinases) such as HCK, the dominant SFK in inflammatory signaling in monocytes/macrophages. The SFKs function upstream to SYK kinases which further phosphorylates VAV1, a guanine nucleotide exchange factor (GEF) that promotes the activation of RHO-GTPases, such as RAC1, by catalyzing the exchange of GDP to GTP, which transforms RAC1 from the inactive to the active-bound state. The GTP activated RAC1 binds PAK1 which induces its activation. This signaling cascade is induced by a range of inflammatory related extracellular ligands including chemokines and cytokines, growth factors such as VEGFR , TREM ligands and FC receptor ligands which induce FC-mediated phagocytosis involving coordinated process of cytoskeleton rearrangement (Turner et al., 2002; Page et al., 2009). The inventors also found these pathways in the disrupted meta-pathway functional enrichment analysis (FIG. 3E, right and biorxiv.org/content/10.1101/2021.06.16.448558v1 .supplementary-material?versioned=true). The latter are also tightly related to ROS and NADPH oxidase activation through the regulation of RAC1 (Zhao et al., 2003). Of note, it has been noted that suppression of VAV1-RAC1-PAK1 signaling, predominately in innate immune cells mediates remission in Crohn's disease (Parikh et al., 2014). Taken together, these observations showcase the power of Disruption Network to identify masked, individual level, signal and suggest that the RAC1-PAK1 signaling cascade, is significantly disrupted in non-responders, during treatment.

### Example 4

### RAC1-PAK1 Cytoskeleton Organization Signaling is Elevated in Responders' Peripheral Monocytes Pre-Treatment

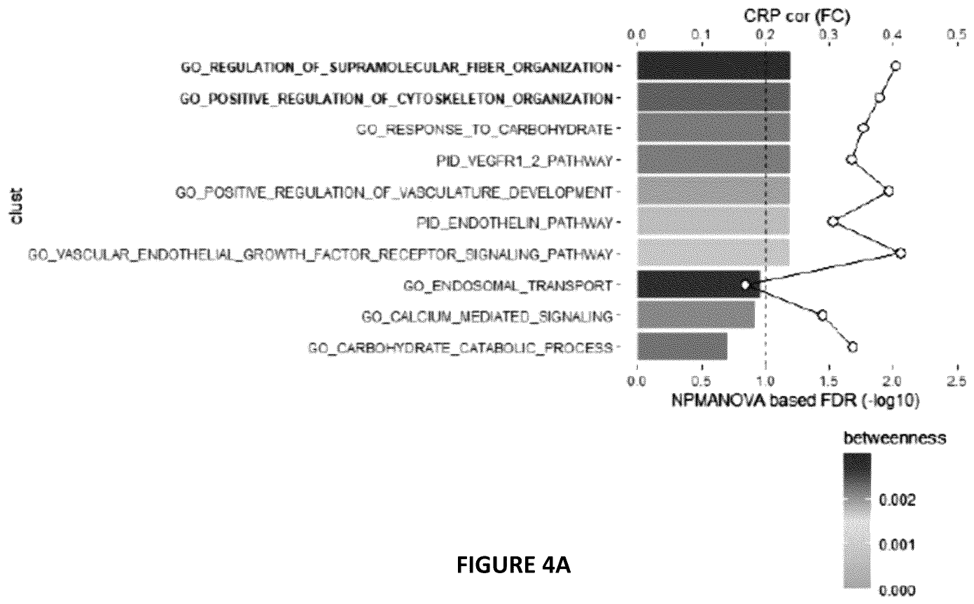

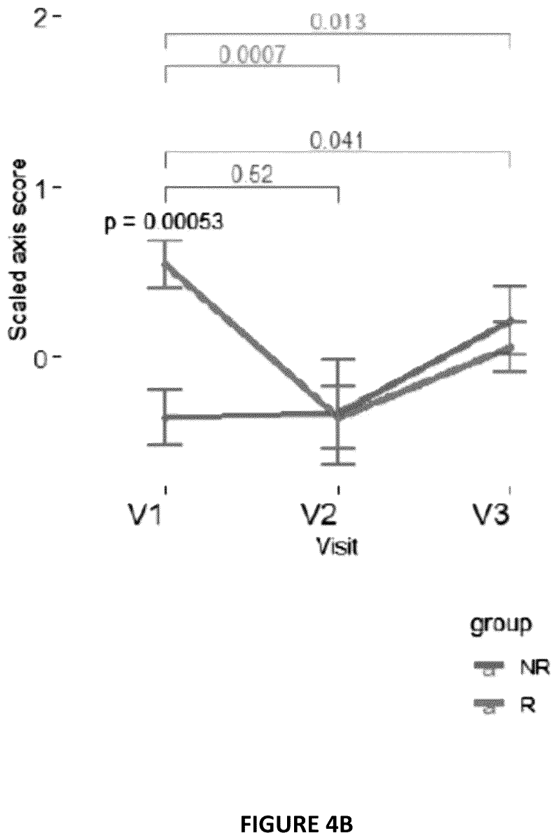

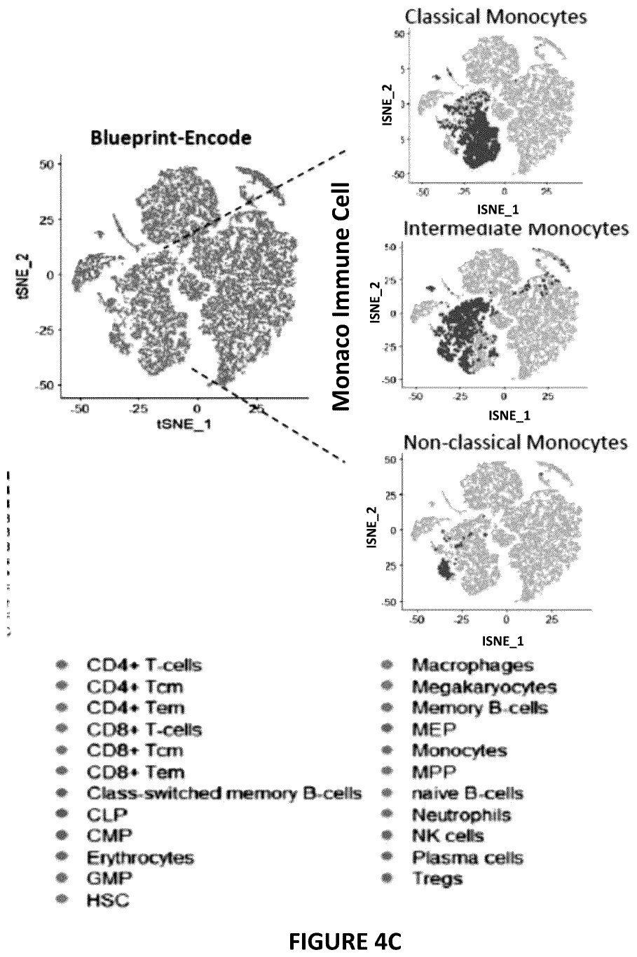

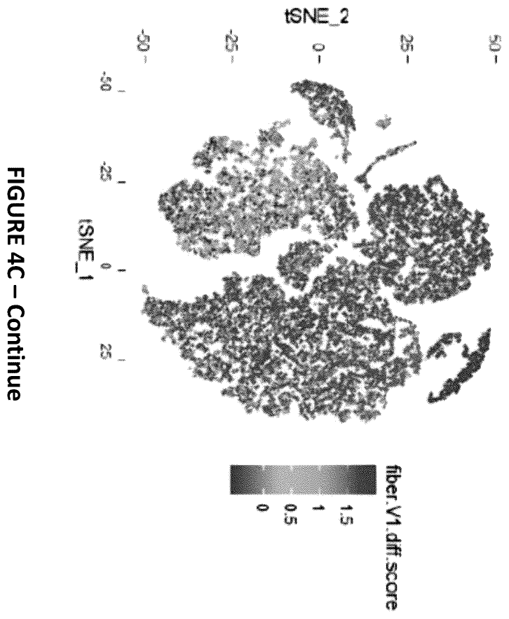

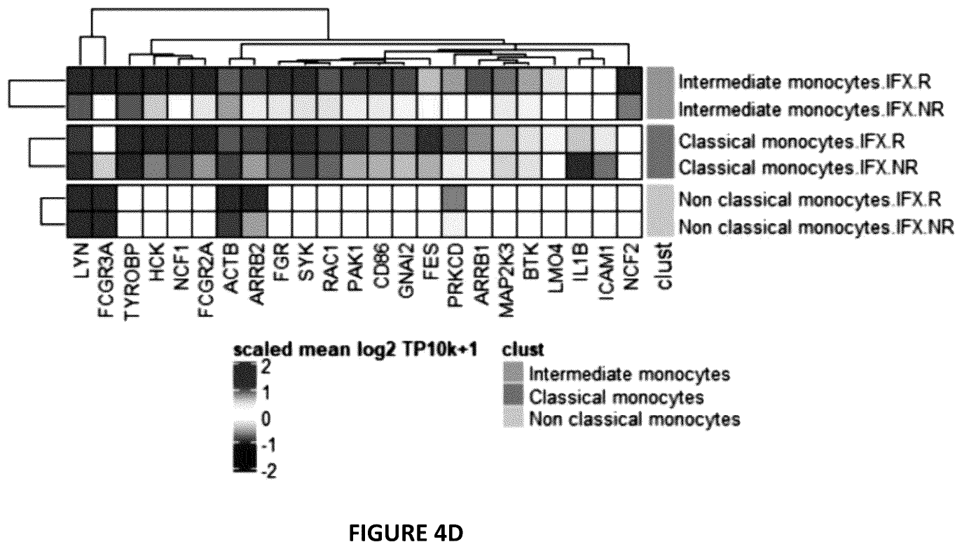

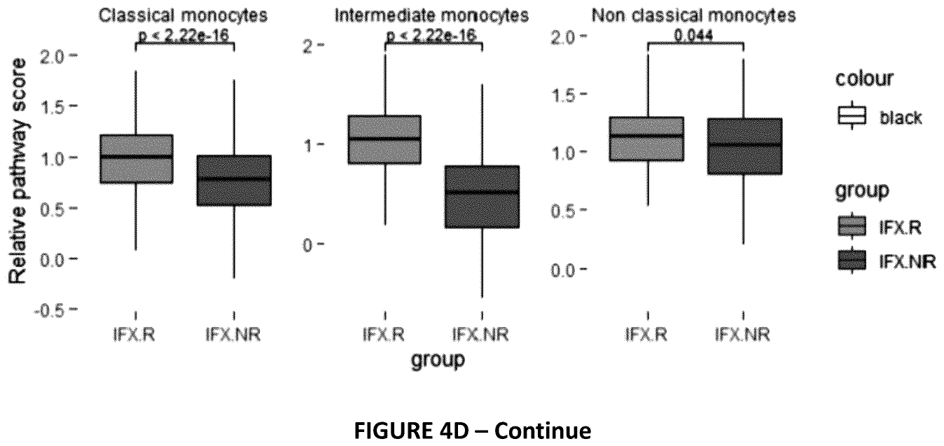

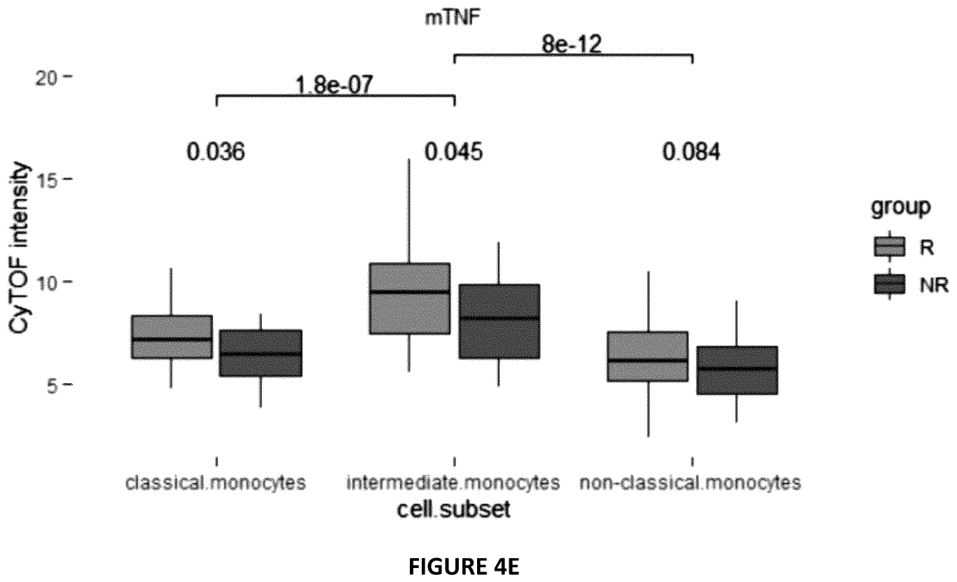

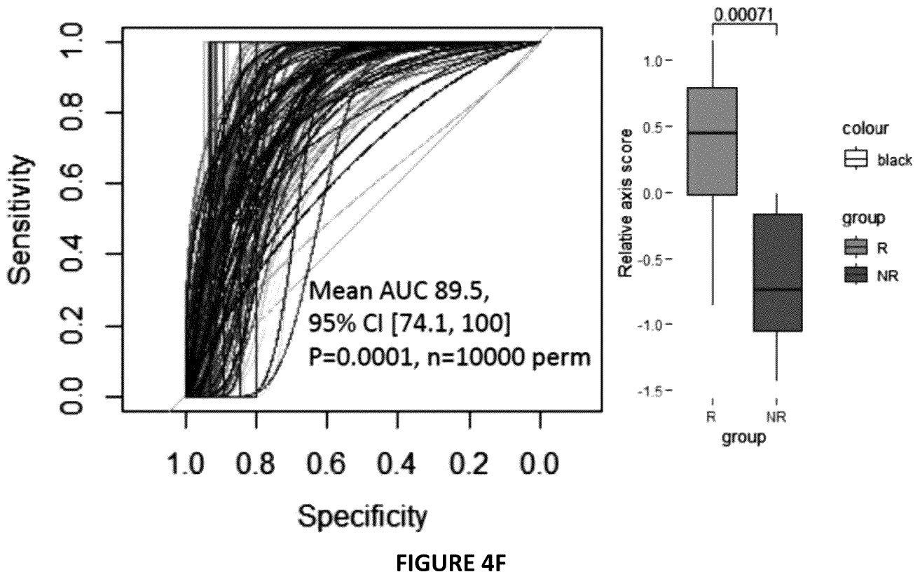

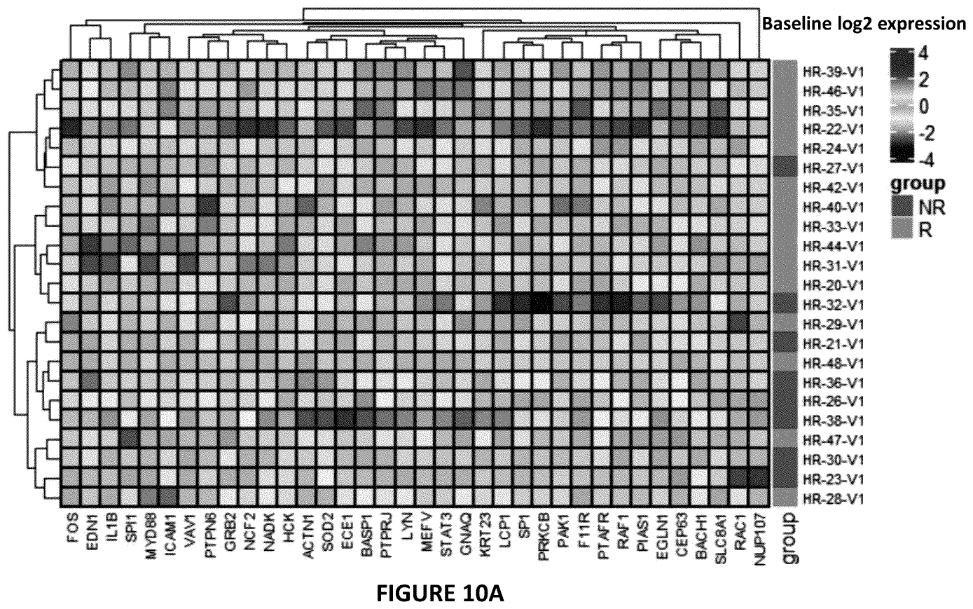

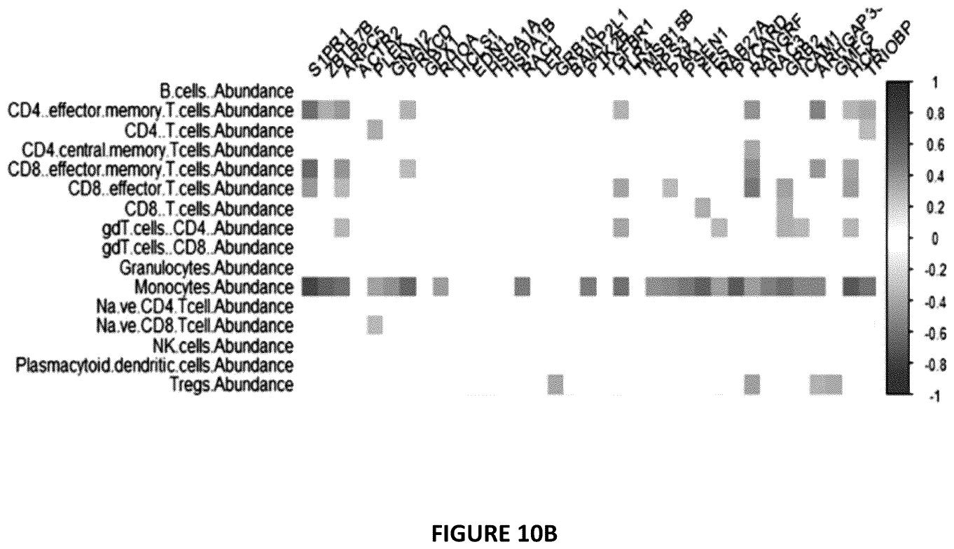

The inventors next asked whether dynamics concomitant differential programs between response groups can be identified pre-treatment. Looking at the feature level, the inventors found that most of the pre-treatment differentially expressed genes were increased in responders, including genes involved in the RAC1-PAK1 axis (FDR<0.1, Wilcoxon test, FIG. 10A). On the pathway level the inventors observed that the fiber organization pathway, presented pre-treatment disparity between the two response groups (FDR<0.1, NPMANOVA) and high correlation with clinical CRP (Spearman's r=0.4, p=0.06), in addition to the high centrality in the response network (FIG. 4A, left and biorxiv.org/content/10.1101/2021.06.16.448558v1. supplementary-material?versioned=true). The relative pathway score of the cytoskeleton-organization signaling was higher in responders pre-treatment compared to non-responders (P<0.0006, one tail Wilcoxon test), and was downregulated following efficient treatment (P<0.001 and P<0.05 for W2 and W14 compared to baseline, one tail Wilcoxon test). This is in contrast to non-responders which showed insignificant dynamics at W2 and even opposite trend in W14 (P=0.52 and P=0.041 for W2 and W14 compared to baseline, one tail Wilcoxon test) (FIG. 4B).

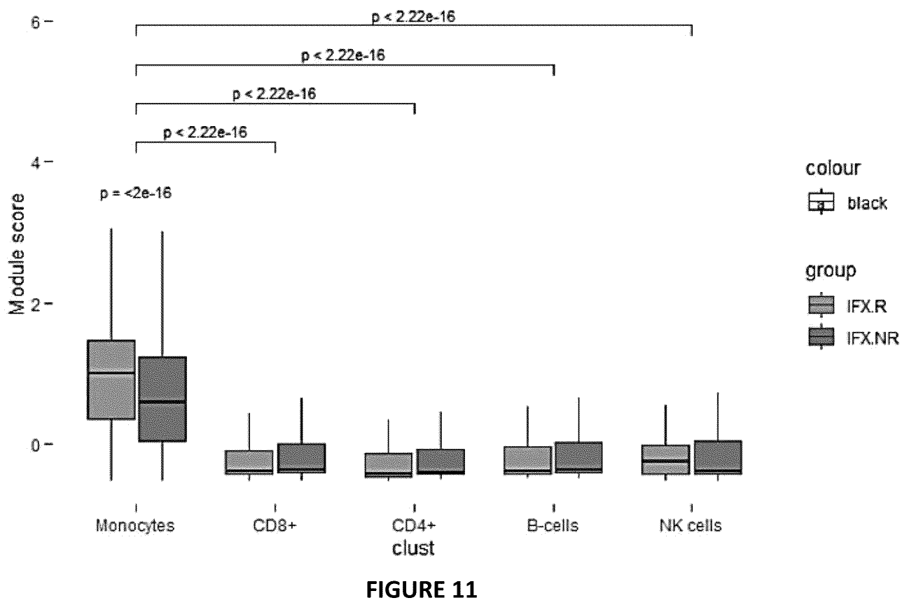

The fiber organization pathway associated with pre-treatment response and treatment dynamics represents distinctive differences in cellular transcriptional states between response groups, rather than differences reflecting cellular composition alterations, as our analyses accounted for variation in major blood cell types. Therefore, the inventors next aimed to dissect the cellular origin of the fiber organization related core genes. First, the inventors tested the correlation between the canonical cellular frequencies as obtained by CyTOF, and the bulk unadjusted expression of the fiber organization related genes (FIG. 10B). The results indicated that the majority of the genes in the target pathway, were positively associated with monocytes abundance. To further validate the cellular origin and the fiber organization related transcriptional cell state in the two response groups, the inventors performed single-cell RNA sequencing (scRNA-seq) using peripheral blood mononuclear cells (PBMCs) from pre-treatment samples of representative responder and non-responder patients (FIG. 4C, left; see Methods). Assessment of the fiber organization related expression in the cellular level, confirmed that monocytes were highly associated with the distinctive pathway expression (P<2.2e-16, for expression in monocytes compared to the other cell types, Wilcoxon test, FIG. 4C, right and FIG. 11A).

Given the significant linkage between monocytes and the differential fiber organization pathway, and to deepen the cell centered information related to the fiber organization pathway, the inventors expanded the fiber organization differential genes through intersection (biorxiv.org/content/10.1101/2021.06.16.448558v1 .supplementary-material?versioned=true) of knowledge based (combined score>900, stringDB, and biorxiv.org/content/10.1101/2021.06.16.448558v1.supplementary-material?versioned=true) and data-driven based (Monocytes single-cell based co-expression network, Spearman's r, thinning percentile: 0.05 and biorxiv.org/content/10.1101/2021.06.16.448558v1 .supplementary-material?versioned=true) (see Methods). Then, the inventors assessed the pathway related expression in monocyte subsets, which were previously shown to exhibit distinct phenotypes and functional properties in health, and in the context of IBD and other related immune mediated diseases such as RA and SLE (Kapellos et al., 2019; Gren and Grip, 2016; Tsukamoto et al, 2017; and Hirose et al., 2019). The results indicated that intermediate monocytes contributed the most to the fiber organization distinctive expression between responders and non-responders, pre-treatment (|FC|=2.13, P<2.2e-16 in intermediate monocytes vs. |FC|=1.3, P<2.2e-16 and |FC|=1.1, P<0.05 in classical and non-classical monocytes respectively by Wilcoxon test, FIG. 4D). Interestingly, the inventors detected significantly increased membrane TNF (mTNF) on intermediate monocytes compared to the other subsets (P<5e-07, one tail Wilcoxon test, FIG. 4E), suggesting these cells serve as drug targets, thereby explaining their tight linkage to drug response.

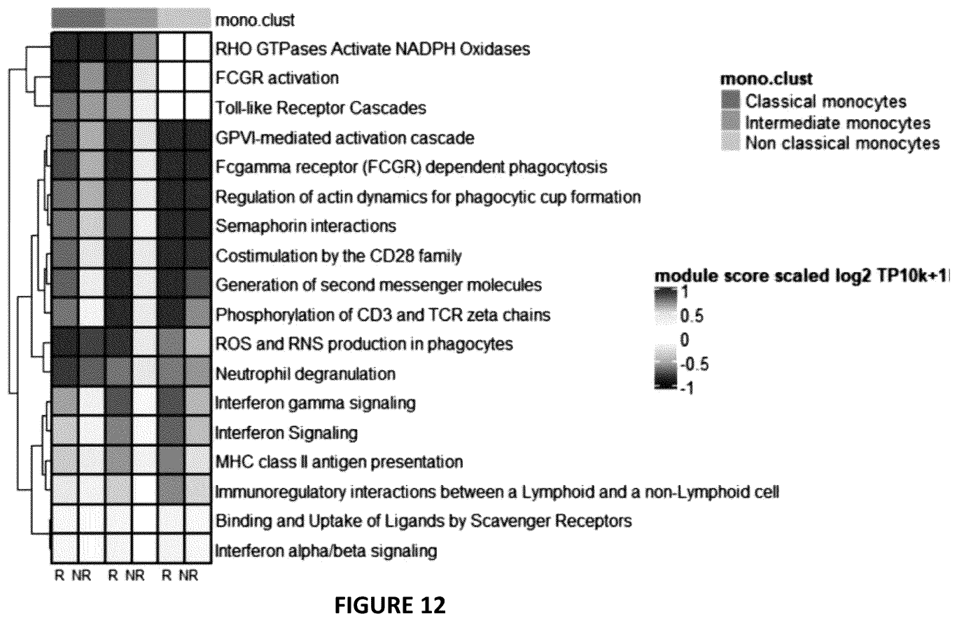

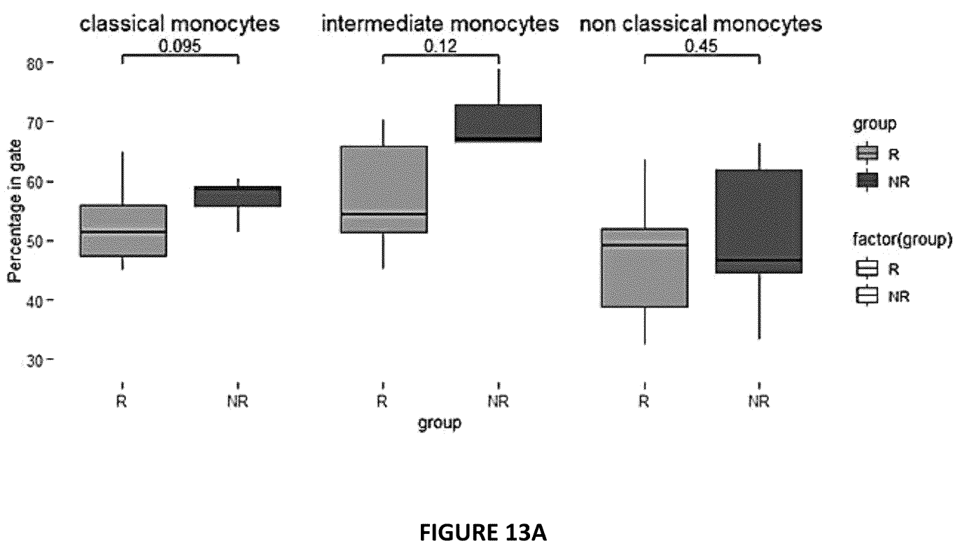

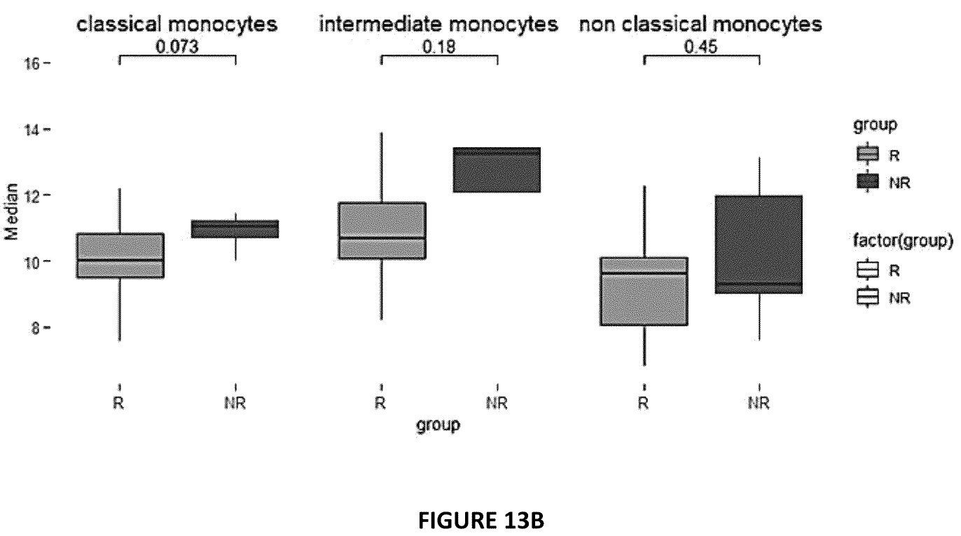

The inventors next tested whether the pre-treatment fiber organization pathway expression could predict treatment resistance based on the extended baseline differential fiber organization related genes (Generalized linear models (GLMs) with elastic net penalization: alpha=0.5, lambda=0.26, 100 repeated 2-fold CV). The inventors observed that the pathway score of a core set of 6 genes (RAC1, PAK1, LYN, ICAM1, IL1B and FCGR3A) could discriminate non-responders from responders at a mean AUC of 89.5%, 95% CI (74.1, 100) (P=0.0001, n=10,000 perm), supporting a common mechanism of non-response to treatment (FIG. 4F). By applying targeted network analysis of the predictive fiber organization pathway in intermediate monocytes scRNA-seq data, the inventors found that the FC-gamma receptor signaling and functionally related pathways including phagocytosis and ROS metabolic processes were highly enriched in the co-expression network and provide the best differentiation between response groups at baseline (FIG. 12 and biorxiv.org/content/10.1101/2021.06.16.448558v1.supplementary-material?versioned=true).

### Example 5

Pre-Treatment RAC1-PAK1 Axis Expression was also Validated as a Predictor for IFX Response Across Diseases in an Additional IBD Cohort and in Three Public RA Cohorts

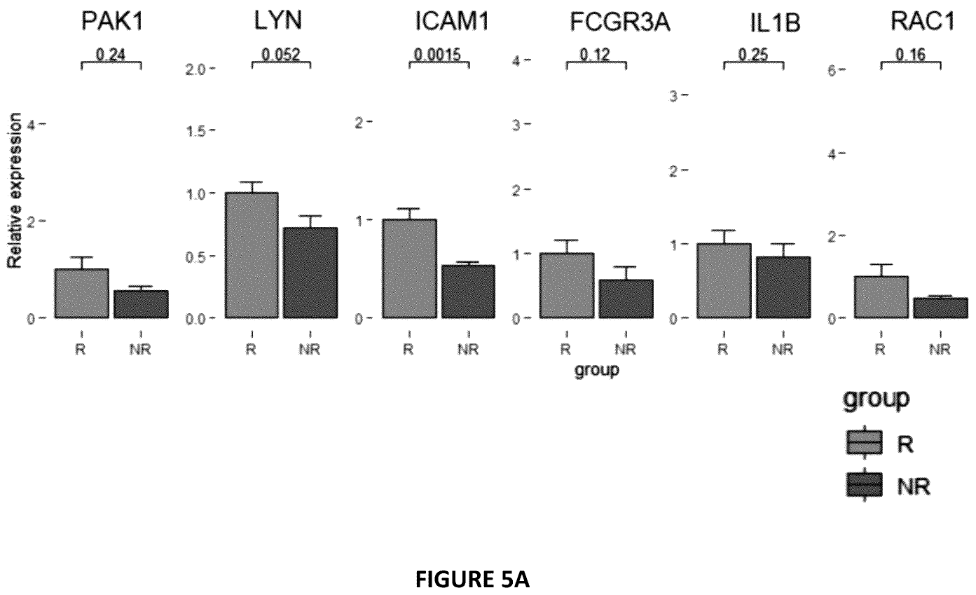

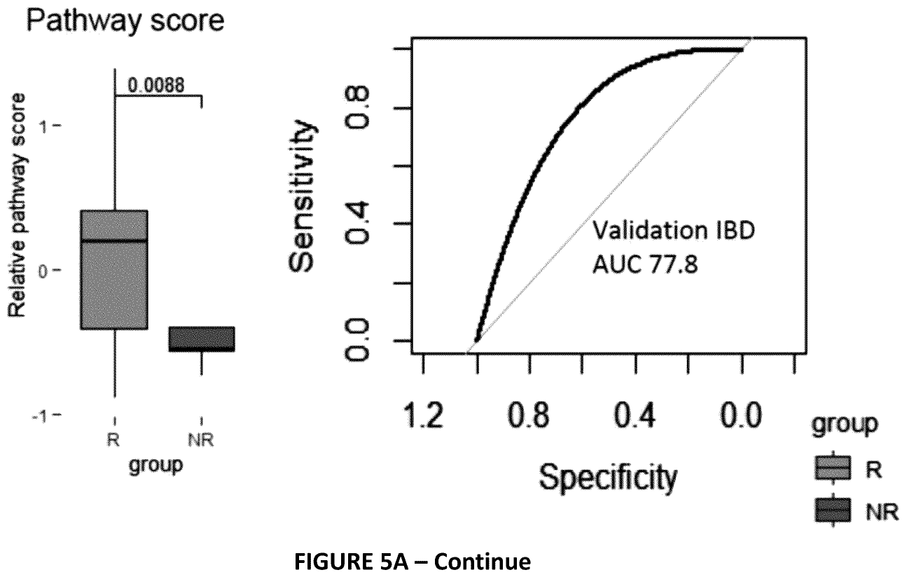

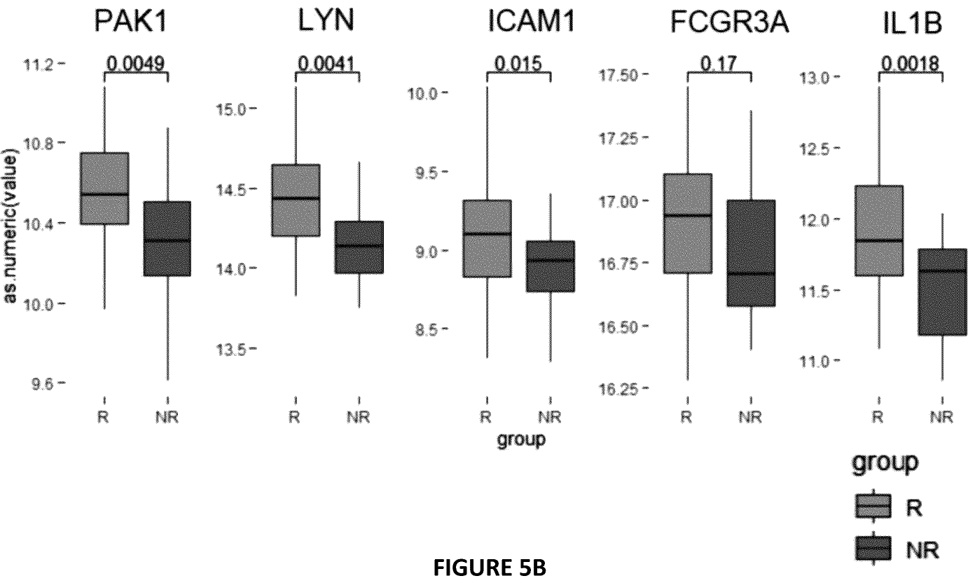

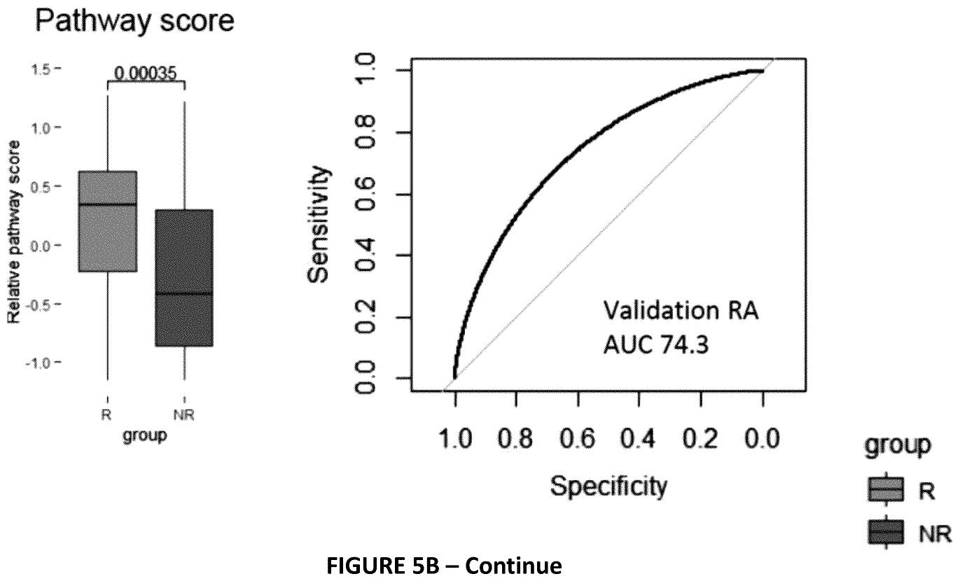

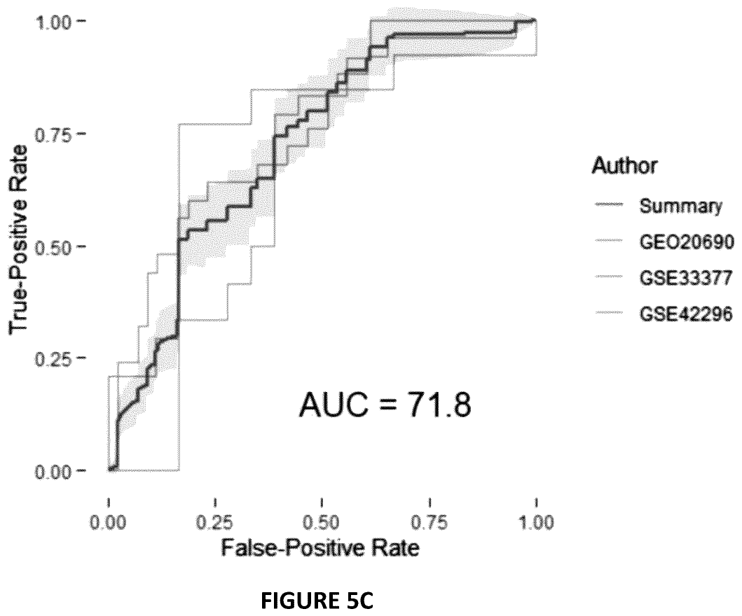

To further validate the herein disclosed findings, the inventors tested an additional independent validation cohort of 20 and 9 responding and non-responding patients, respectively, which were naive to biological treatment and were treated with thiopurines or steroids only as a co-therapy (see biorxiv.org/content/10.1101/2021.06.16.448558v1.supplementary-material?versioned=true). The results indicated that the pre-treatment RAC1-PAK1 axis, was differentially expressed between responders and non-responders in the validation cohort (Fisher's combined P<0.005 & relative pathway score P<0.005, T test) as well, supporting the primary findings thereby demonstrating that pre-treatment reduced expression of the RAC1-PAK1 axis is associated with non-response (AUC of 77.8%; FIG. 5A).

To assess the relevance of the PAK1-RAC1 axis to anti-TNF resistance prediction in other related diseases, the inventors tested publicly available datasets of whole blood samples from RA patients, pre- IFX treatment (GSE2069035, GSE3337736, GEO4229621). Gene expression was adjusted to major cell type contributions, which were evaluated by deconvolution (see Methods). The results confirmed the increased expression of the axis related genes in RA responders, pre-treatment (Representative cohort GEO20690, FIG. 5B). Application of fiber organization predictive signature to the pre-treatment multiple RA cohorts was able to separate anti-TNF responders and non-responders (Meta ROC AUC=71.8, FIG. 5C). These findings expand the predictive value of the RAC1-PAK1 axis to other IFX-treated related diseases such as RA. Taken together, these observations demonstrate that baseline RAC1-PAK1 axis status in monocytes, which is a final common pathway of several proximal immunoreceptor signaling routes, differentiates response groups and ultimately impacts response potential across diseases.

### Discussion

Despite substantial inter-individual heterogeneity, most studies focusing on anti-TNF response and resistance are population based, and thus limited in their ability to monitor individual variation in drug response. In addition, although blood-based biomarkers offer important advantages as response diagnostic tools in terms of cost effectiveness and availability, they introduce high background noise and inter-individual variations. The relatively small cohort sizes also poses analytical challenge associated with low statistical power and robustness. To address these challenges, the inventors devised ‘disruption networks’, a cell-centered personalized computational framework which exposes a finer resolution of differences between patient groups, to identify blood-based individual-specific patterns of non-response during treatment and at baseline, otherwise missed by standard analysis. Using this methodology, the inventors could detect changes not only in the feature expression level but also alternations related to cross feature responses reflecting expression dysregulation. Aggregation to pathway level disruption estimates further provide functional context. By using this framework, the inventors provide a systematic dissection of effective response dynamics following IFX treatment and identification of functional paths deviating from normal response in non-responders, which enable revealing common determinants associated with drug resistance across IFX treated immune mediated diseases including IBD and RA.

Although TNF is a pleiotropic cytokine that mediates inflammation through multiple ways affecting both innate and adaptive immune system (Billiet et al., 2014; and Kalliolias and Ivashkiv, 2016), the inventors found that most of the observed early normal response alternations following IFX treatment were related to innate immune pathways. Among the normal downregulated response pathways, the inventors found the expected downregulation of NF-kB and TNF signaling via NF-KB and MAPK activation, together with pathways related to TLR and FCGR signaling. This observation is in line with previous studies reporting SNPs association with anti-TNF responsiveness in the following genes: TLR2, TLR9, and MAP3K14, TNFRSF1A (TNFR1) and TNFAIP3 or cytokines regulated by NFκKB including IL1B, IL1RN, IL6, and IFNG, and FCGR polymorphism (Louis et al., 2004; Linares-Pineda et al., 2018; Louis et al., 2006; and Moroi et al., 2013). The normal IFX response affected pathways were predominantly associated with monocytes function. These results, together with the significant change in monocytes abundance, which also correlated with CRP, suggest that monocytes are a major component mediating the early drug response/non-response in IFX treatment. A decreased frequency of monocytes was previously reported in anti-TNF treated IBD (Shen et al., 2005; Shen et al., 2005; and Lügering et al., 2001) and RA (Batko et al., 2019) patients, further supporting the observed decrease in monocyte proportions following treatment in our real-life cohort. Monocytes importance for IFX response was demonstrated by previous studies showing that the anti-proliferative and cell-activation suppression therapeutic effect of IFX on T-cells was completely dependent on the presence of FC-expressing monocytes in a mixed lymphocyte reaction, an effect that was abrogated when isolated T cell cultures were used (Rossol et al., 2007; and Digby-Bell et al., 2020). In addition, granulocyte/monocyte adsorption (GMA) treatment which regained long-term response after experiencing loss of response during IFX treatment further corroborate the herein disclosed findings (Yokoyama et al., 2018; Fukunaga, K. et al., 2010; and Yokoyama et al., 2020). Taken together, these results support the potential for subset specific targeted combination therapy to overcome anti-TNF resistance in the future (Sedykh et al., 2018; and Zhao, 2020).

Given the characterized global normal anti-TNF response, the herein disclosed ‘disruption networks’ framework, indicated that the cytoskeleton organization pathway, and particularly the RAC1-PAK1 signaling axis, which is among the central pathways associated with normal response, exhibited disrupted dynamics in non-responders and its expression at baseline was predictive of treatment response. The RAC1-PAK1 axis is a final common pathway shared by several proximal immunoreceptors, controlling for actin cytoskeletal movement and activation of the respiratory burst and phagocytic activity in innate cells. RAC1 was identified as a susceptibility gene for IBD (Muise et al., 2015), and TNF was previously shown to potently stimulate p21Rac1 GTP loading, supporting antagonism of this effect by anti-TNF. The pathway's relevance for IBD pathophysiology and recovery response, was further reinforced by the effect of thiopurines, which were shown to inhibit RAC1 activation (Seinen et al., 2016), thereby promoting remission in Crohn's disease. Thiopurines were shown to reverse the cytoskeletal aberrations and restore normal phagocytic function in monocytes (Parikh et al., 2014; and Jing et al., 2018) further corroborating the association of RAC1-PAK1 axis expression in monocytes as central for IFX response observed in this study. This observation is also consistent with the fact that combination therapy of anti-TNF is superior to anti-TNF monotherapy (Lim and Chua, 2018; and Colombel et al., 2010), and suggests that this effect is mediated not only by controlling anti-drug antibody (ADA) levels, but conceivably also by the induction of a mutual effect on RAC1 suppression in monocytes. Interestingly, by applying the disruption network approach, TREM1 adaptor (TYROBP/DAP12), which was previously reported by us as a biomarker associated with anti-TNF response (Gaujoux, et al., 2019), was detected in the expanded differential fiber organization signature. This biomarker was highly co-expressed with the RAC1-PAK1 axis in the monocyte specific scRNA data, and is also functionally related through shared signaling factors, implicating disruption networks as an efficient computational approach in constituting a meaningful biological signal in relatively small heterogeneous cohorts. The monocytes single cell-based co-expression network, of the pre-treatment differential RAC1-PAK1 axis, indicated a distinctive expression, primarily in intermediate monocytes subset, which was related to FcyR dependent phagocytosis and interferon signaling that among other functions induces FcyR responses and affects ROS production. The RAC1-PAK1 axis is a downstream FCYR signaling pathway. The relevance of FcR-mediated therapeutic outcomes of anti-TNF in IBD was implicated by previous studies both in mouse models and humans (Chen et al, 2020). It was shown that low FcγR affinity IgG4 anti-TNF did not reach clinical efficiency. This was also demonstrated using a colitis mouse model, in which administration of low FcγR affinity anti-TNF abolished therapeutic response (Chen et al., 2014). Furthermore, FCYR3A polymorphism, which increased FcγR affinity was shown to better affect the treatment response to infliximab in Crohn's disease (Louis et al., 2004; and Moroi et al, 2013). The last was further supported by our findings showing its high relevance in the predictive response signature.

Interestingly, the inventors found that the RAC1-PAK1 axis was predictive of anti-TNF responsiveness also in RA, which further provides additional validation for the signature predictivity and supports a common baseline elements that contribute to response across anti-TNF treated immune mediated diseases. Similarly to IBD, there are also studies in RA which provide evidence for the linkage of the RAC1-PAK1 upstream FcγR to disease susceptibility, demonstrating association of FcγR3 polymorphism with increased risk for RA (Chen et al., 2020; Chen et al., 2014; and Morgan et al., 2005). The FcγR3 polymorphism was associated with a failure in FcγR-mediated clearance of immune IgG-containing complexes, which may be of high relevance also with regard to therapeutic antibodies such as Infliximab. Generally, FcγR3A is known as a key receptor for monocytes effector response including phagocytosis and ADCC. Monocytes responding to antibody-coated targets, were shown to contribute to clearance and potentially to therapeutic efficacy (Roberts et al., 2020). Furthermore, it was shown that increasing the IgG binding affinity by monocyte CD16a increased ADCC (Herter et al., 2014). These results further suggest that a common element of enhanced RAC1-PAK1 signaling through increased expression or affinity for FcγR3A expressed on monocytes/macrophage across immune mediated diseases treated with anti-TNF, may enhance clinical efficacy. The documented differences in anti-TNF blockers effects between diseases as IBD and RA, might explain the slight reduced performance of the predictive signature in RA compared to IBD. The relevance of the RAC1-PAK1 axis and the upstream FcγR to anti-TNF response in additional immune related diseases remains to be determined.

Given the massive volume of inter-individual heterogeneous genomic data, the inventors expect that the ‘disruption networks’ personalized computational framework disclosed herein, will provide a robust and efficient analytical tool to leverage this information in order to develop blood-based diagnostics to a broad panel of drugs in IBD, which will enable increasing response rates by a personalized treatment through the identification of patients with a high chance to respond to a given medication.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1.-9. (canceled)

10. A method for treating a subject afflicted with a TNFα related disease, comprising the steps:
a. determining whether an expression level of at least one gene involved in the RAC1-PAK1 axis is increased above a pre-determined threshold, in a sample obtained or derived from said subject; and
b. administering to said subject determined as having increased expression level of at least one gene involved in the RAC1-PAK1 axis above a pre-determined threshold, a therapeutically effective amount of a TNFα inhibitor, wherein said TNFα inhibitor is an anti-TNFα antibody or a TNFα mimicking receptor,
thereby treating the subject afflicted with said TNFα related disease.

11. The method of claim 10, wherein said administering comprises intravenously administering or subcutaneously administering.

12. The method of claim 10 , further comprising a step (c) comprising at least once determining the expression level of said at least one gene involved in the RAC1-PAK1 axis in the administered subject, wherein an increase in the expression level of said at least one gene involved in the RAC1-PAK1 axis above a pre-determined threshold is indicative of said administered subject being responsive to said treatment with said TNFα inhibitor.

13. The method of claim 10, wherein said at least one gene involved in the RAC1-PAK1 axis is selected from the group consisting of: PAK1, ICAM1, FCGR3A, LYN, IL1B, RAC1, and any combination thereof.

14. The method of claim 10, wherein said TNFα inhibitor is selected from the group consisting of: Infliximab, Adalimumab, Golimumab, Certolizumab pegol, and Etanercept.

15. The method of claim 10, wherein said TNFα related disease is selected from the group consisting of: Crohn's disease, ulcerative colitis, rheumatoid arthritis, juvenile idiopathic arthritis, polyarticular juvenile idiopathic arthritis, psoriasis, psoriatic arthritis, plaque psoriasis, ankylosing spondylitis, hidradenitis suppurativa, uveitis and any combination thereof.

16. The method of claim 10, wherein said sample obtained or derived from said subject comprises RNA molecule, protein, or both.

17. The method of claim 16, wherein any one of said: RNA molecule, protein, and both, is extracted from blood or a biopsy derived or obtained from said subject.

18. The method of claim 10, further comprising a step preceding step (a), comprising extracting RNA, protein, or both, from blood or a biopsy derived or obtained from said subject.

19. The method of claim 10, wherein said determining based on said RNA comprises: hybridization, amplification, sequencing, or any combination thereof, of said RNA.

20. The method of claim 10, wherein said determining based on said protein is by an immune assay comprising an antibody having increased binding affinity to said protein.

21. The method of claim 10, wherein said increase is at least a 10% increase.

22. A kit for determining the suitability of a subject for a treatment with a tumor necrosis factor alpha (TNFα) inhibitor, wherein said TNFα inhibitor is an anti-TNFα antibody or a TNFα mimicking receptor, the kit comprising any one of:
a. at least one oligonucleotide comprising a nucleic acid sequence capable of hybridizing to at least one transcript of said at least one gene involved in the RAC1-PAK1 axis, or a complementary DNA thereto;
b. at least one antagonist having specific binding affinity to a protein product of said at least one gene involved in the RAC1-PAK1 axis; and
c. any combination of (a) and (b).

23. The kit of claim 22, wherein said at least one gene involved in the RAC1-PAK1 axis is selected from the group consisting of: PAK1, ICAM1, FCGR3A, LYN, IL1B, RAC1, and any combination thereof.

24. The kit of claim 22, wherein said at least one oligonucleotide comprises any one of a probing oligonucleotide, a pair of primers capable of amplifying a complementary DNA of said at least one transcript of said at least one gene involved in the RAC1-PAK1 axis in a polymerase chain reaction (PCR), and both.

25. The kit of claim 22, wherein said pair of primers is selected from the group consisting of SEQ ID Nos: 1-14.

26. The kit of claim 22, wherein said antagonist comprises an antibody.


SEQUENCE LISTING (14 sequences)

SEQ ID NO: 1 - DNA, 22 bp, Synthetic construct
  source 1..22: mol_type = other DNA
agcggctgacgtgtgcagtaat

SEQ ID NO: 2 - DNA, 24 bp, Synthetic construct
  source 1..24: mol_type = other DNA
cgaggggctgagacatttacaaca

SEQ ID NO: 3 - DNA, 24 bp, Synthetic construct
  source 1..24: mol_type = other DNA
agtttcagaagatgaggatgatga

SEQ ID NO: 4 - DNA, 23 bp, Synthetic construct
  source 1..23: mol_type = other DNA
aatcacagaccgtgtgtatacag

SEQ ID NO: 5 - DNA, 23 bp, Synthetic construct
  source 1..23: mol_type = other DNA
gctggatttcctgaagagcgatg

SEQ ID NO: 6 - DNA, 23 bp, Synthetic construct
  source 1..23: mol_type = other DNA
cggtgaatgtagttcttccgctc

SEQ ID NO: 7 - DNA, 22 bp, Synthetic construct
  source 1..22: mol_type = other DNA
ggtgacttgtccactccagtgt

SEQ ID NO: 8 - DNA, 22 bp, Synthetic construct
  source 1..22: mol_type = other DNA
accattgaggctccaggaacac

SEQ ID NO: 9 - DNA, 22 bp, Synthetic construct
  source 1..22: mol_type = other DNA
agcggctgacgtgtgcagtaat

SEQ ID NO: 10 - DNA, 22 bp, Synthetic construct
  source 1..22: mol_type = other DNA
tctgagacctctggcttcgtca

SEQ ID NO: 11 - DNA, 22 bp, Synthetic construct
  source 1..22: mol_type = other DNA
ccacagaccttccaggagaatg

SEQ ID NO: 12 - DNA, 23 bp, Synthetic construct
  source 1..23: mol_type = other DNA
gtgcagttcagtgatcgtacagg

SEQ ID NO: 13 - DNA, 22 bp, Synthetic construct
  source 1..22: mol_type = other DNA
ctggaacaggtgcctaaaggac

SEQ ID NO: 14 - DNA, 22 bp, Synthetic construct
  source 1..22: mol_type = other DNA
gtccagtgtcaggttatccacc